(19) United States
(12) Patent Application Publication
(10) Pub. No.: US 2024/0058431 A1
(43) Pub. Date: Feb. 22, 2024

(54) NOVEL COMPOSITION

(71) Applicant: The University of Liverpool, Liverpool (GB)

(72) Inventors: Aras Kadioglu, West Kirby (GB); Marie Yang, Lurgan (GB); Stavros Panagiotou, Liverpool (GB)

(21) Appl. No.: 18/270,947

(22) PCT Filed: Jan. 5, 2022
(86) PCT No.: PCT/GB2022/050003

§ 371 Date: Jul. 5, 2023

(30) Foreign Application Priority Data
Jan. 5, 2021 (GB) 2100097.1

(51) Int. Cl. A61K 39/09; A61K 39/39; A61P 37/04; A61P 31/04

(52) U.S. Cl. CPC A61K 39/092; A61K 39/39; A61P 37/04; A61P 31/04; A61K 2039/55522

(57) ABSTRACT

There is provided an immunogenic composition comprising at least two antigenic determinants, wherein the antigenic determinants are derived from at least two proteins selected from ABC-T, PavA and ZmpB of a Streptococcus pneumoniae bacterium; an immunogenic composition comprising a genetic construct or constructs encoding at least two antigenic determinants, wherein the antigenic determinants are derived from at least two proteins selected from ABC-T, PavA and ZmpB of a Streptococcus pneumoniae bacterium; and an immunogenic composition comprising at least one antigenic determinant and a genetic construct or constructs encoding at least one different antigenic determinant, wherein the antigenic determinants are derived from at least two proteins selected from ABC-T, PavA and ZmpB of a Streptococcus pneumoniae bacterium. There are also provided vaccines, methods of treating or preventing or immunising against Streptococcus pneumoniae bacterium infections and kits comprising immunogenic compositions.

The present invention relates to an immunogenic composition comprising Streptococcus pneumoniae proteins, a new vaccine that is effective against the Streptococcus pneumoniae bacterium, the invention also comprises methods of treating or preventing a Streptococcus pneumoniae bacterium infection and methods of immunising against Streptococcus pneumoniae bacterial infections. Further included within the scope of the invention are nucleic acids encoding the Streptococcus pneumoniae proteins for inclusion in DNA/RNA-based vaccination.

### BACKGROUND

Streptococcus pneumoniae is a nasopharyngeal commensal bacterium that can also invade normally sterile sites to cause pneumonia, bloodstream infection and meningitis, and also cause non-invasive diseases such as otitis media. Although the pneumococcal population structure and evolutionary genetics are well defined, it is not clear whether pneumococci that cause invasive diseases are genetically distinct from those that do not. Kulohoma et al (Infection and Immunity; 2015, 83, 10, 4165-4896) reported whole-genome sequencing of 140 isolates of S. pneumoniae recovered from bloodstream infection and meningitis and found that these isolates shared a core of 1,427 genes and that there was no difference in the core genome or accessory gene content from these disease manifestations. Gene presence/absence alone therefore does not explain the invasive behaviour of pneumococci, but that instead, genetic variations such as nucleotide polymorphisms or unitigs, may be the contributory factors.

It is known from the prior art that there are more than 100 pneumococcal serotypes, each of which produce a biochemically distinct capsular polysaccharide (CPS) and vary in propensity to cause invasive disease. Serotype 1 is one of the most common causes of invasive pulmonary disease (IPD) worldwide accounting for 11.7% of cases in Africa and in contrast to other serotypes, serotype 1 is associated with outbreaks in closed communities and lethal meningitis outbreaks in West Africa. The high burden of serotype 1 in IPD emphasizes the need for an effective vaccine against this serotype. Cornick et al; (Vaccine, 2017, 35(6), 972-980) showed the prevalence of seven protein vaccine candidates (CbpA, PcpA, PhtD, PspA, SP0148, SP1912, SP2108) among 445 serotype 1 pneumococci from 26 different countries, across four continents. CbpA (76%), PspA (68%), PhtD (28%), PcpA (11%) were not universally encoded in the study population, and would not provide full coverage against serotype 1. For example, although PcpA was widely present in the European cases (82%), it was rarely present in the African cases (2%). They proposed that a multi-valent vaccine incorporating CbpA, PcpA, PhtD and PspA could be predicted to provide coverage against 86% of the global population and advocated further investigation into a protein vaccine additionally incorporating a single variant of SP0148, SP1912 and/or SP2108 from Streptococcus pneumoniae TIGR4.

Pneumococcal vaccines protect against serious and potentially fatal pneumococcal infections in high-risk individuals such as infants under two years of age, adults over 65 years old and children and immunocompromised individuals with certain long-term health conditions such as heart and kidney conditions. Two types of pneumococcal vaccine are currently generally used in the UK and, depending on age, they are differentially administered to infants and those over 65 years of age. Prevenar 13® (also known as PCV-13) comprises a pneumococcal polysaccharide conjugate vaccine (PCV) that provides protection against 13 serotypes of Streptococcus pneumoniae bacterium and is used to vaccinate children under two years of age, whereas the pneumococcal polysaccharide vaccine PPV-23 (also known as Pneumovax) which protects against 23 serotypes is given to people of 65 years and over. A problem with this vaccine is that it is not particularly effective in the under twos. Indeed, both vaccines are thought to be only between 50-70% effective at preventing pneumococcal disease. Furthermore, a major limitation of PCVs is they only elicit protective antibodies against the serotypes included in the vaccine formulation. As a result, non-vaccine serotypes can increase in frequency in IPD and carriage post-PCV introduction, as observed following PCV7 introduction in the USA (Weinberger et al; Lancet, 2011, 378; 1968-1973).

Pre-requisites for an efficacious pneumococcal vaccine are conserved expression and minimal variation of the target antigen in the pneumococcal population, and the capability to induce a robust human immune response.

A further desire is to provide a pneumococcal vaccine that is effectual across all ages and in particular, neonates and infants.

Demonstrated herein is an efficacious pneumococcal vaccine with a strongly suggested capability of inducing a robust human immune response. This vaccination will be effectual across all ages and in particular, neonates, infants, the elderly and immunocompromised individuals.

The 100+ different strains of S. pneumoniae are a major, global, cause of morbidity and mortality in children and adults. Marketed pneumococcal vaccines such as Prevenar-13 (children) and Pneumovax-23 (adults), are widely used, however, they use strain-specific carbohydrate antigens making them expensive to manufacture (the world's most expensive vaccines, pre-Covid pandemic) and dramatically limiting their coverage. The present invention was developed in a completely different manner to existing pneumococcal vaccines: instead of targeting the sugar molecules on the outside of the bacteria—which vary from strain to strain, thus limiting the range of protection, the present invention was developed with the intention of targeting highly conserved proteins that are present on all 100 strains of pneumococcus. Our studies have shown that immunological compositions of the present invention are capable of inducing an equivalent or better protective immune response against pneumococcal strains covered by Pfizer's PCV-13 vaccine, as well as strains that are not covered. The combination of broad coverage and low manufacturing costs differentiates the immunological compositions of the present invention from all existing and development stage vaccines against pneumococcus.

### BRIEF SUMMARY OF THE DISCLOSURE

According to a first aspect of the invention there is provided an immunogenic composition comprising at least two antigenic determinants, wherein the antigenic determinants are derived from at least two proteins selected from ABC-T, PavA and ZmpB of a Streptococcus pneumoniae bacterium.

According to a second aspect of the invention there is provided an immunogenic composition comprising a genetic construct or constructs encoding at least two antigenic determinants, wherein the antigenic determinants are derived from at least two proteins selected from ABC-T, PavA and ZmpB of a Streptococcus pneumoniae bacterium.

According to a third aspect of the invention there is provided an immunogenic composition comprising at least one antigenic determinant and a genetic construct or constructs encoding at least one different antigenic determinant, wherein the antigenic determinants are derived from at least two proteins selected from ABC-T, PavA and ZmpB of a Streptococcus pneumoniae bacterium.

The present invention also provides a vaccine comprising an immunogenic composition according to the first, second or third aspects of the invention.

The present invention further provides a method of treating, preventing, diagnosing or screening for a Streptococcus pneumoniae bacterium infection in an individual comprising administering a sufficient amount of the immunological composition of the first, second or third aspects of the invention or a vaccine of the invention to treat, prevent, diagnose or screen for the Streptococcus pneumoniae bacterium infection in an individual in need thereof.

The present invention further provides a method of immunising against a Streptococcus pneumoniae bacterium-initiated infection in an individual comprising administering an effective amount of the immunological composition of the first, second or third aspect of the invention or a vaccine of the invention to an individual in need thereof.

The present invention is based on the finding that immunisation with a combination of antigenic determinants derived from any two of the proteins ABC-T, PavA and ZmpB of a Streptococcus pneumoniae bacterium provides a good immunogenic response. The antigenic determinants may be used directly, or they may be administered in the form of a genetic construct encoding the antigenic determinant, and a mixture of antigenic determinants and genetic constructs encoding different antigenic determinants may also be used. Thus, the immunogenic compositions of the present invention may comprise at least two antigenic determinants, or one or more genetic constructs encoding at least two antigenic determinants, or at least one antigenic determinant and at least one genetic construct encoding at least one different antigenic determinant.

In preferred embodiments of the present invention, the immunogenic composition is based on the provision of antigenic determinants derived from each of the proteins ABC-T, PavA and ZmpB of a Streptococcus pneumoniae bacterium, and these may be provided in any combination of antigenic determinants and genetic constructs encoding one or more of the antigenic determinants (for example, there may be three antigenic determinants, two antigenic determinants and one genetic construct encoding a third antigenic determinant, one antigenic determinant and a genetic construct or constructs encoding the second and third antigenic determinants, or a genetic construct or constructs encoding all three antigenic determinants).

In embodiments of the invention in which two or more antigenic determinants are encoded by genetic constructs, each different antigenic determinant may be encoded by a different genetic construct, or two or more different antigenic determinants may be encoded by a single genetic construct. For example, in an immunogenic composition of the present invention comprising a genetic construct or constructs encoding antigenic determinants derived from each of the proteins ABC-T, PavA and ZmpB of a Streptococcus pneumoniae bacterium, the antigenic determinants could each be encoded by a separate genetic construct, or two antigenic determinants could be encoded by one genetic construct and the other encoded by a second genetic construct, or all three antigenic determinants could be encoded by a single genetic construct.

The genetic constructs suitable for use in the present invention comprise any nucleic acid-based structures that are suitable for expressing the antigenic determinants when administered to a recipient. Suitable genetic constructs include DNA constructs (for example, a DNA plasmid or DNA vaccine), RNA constructs (for example, an RNA vaccine or messenger RNA), bacterial-based constructs and viral-based constructs (such as inactivated or live-attenuated virus constructs).

DNA vaccines contain DNA that codes for specific proteins (antigens) from a pathogen. The DNA is injected into the body and taken up by cells, whose normal metabolic processes synthesize proteins based on the genetic code in the plasmid that they have taken up.

RNA vaccines or mRNA (messenger RNA) vaccines are a type of vaccine that uses a man-made copy of a natural chemical to produce an immune response. The vaccine transfects molecules of synthetic RNA into human cells and stimulates an adaptive immune response, typically the mRNA molecule is coated with a drug delivery vehicle, usually PEGylated lipid nanoparticles.

The present invention also includes the possibility of using DNA/RNA using delivery systems such as live attenuated viruses, such as for example and without limitation, retrovirus, adenovirus, herpes simplex virus, vaccina virus or liposome-based delivery systems, for example emulsions, microparticles, immune-stimulating complexes ISCOMs and liposomes

The immunogenic composition of the present invention may be for human usage in human medicine. Preferably the composition is for administration to a subject. Preferably the subject is human. In one embodiment, the subject is an adult such as an elderly adult greater than 65 years old; an infant less than 1 year old; a toddler between 1 and 2 years old; a young child between 2 and 5 years old; or an immunocompromised individual of any age.

In an embodiment of the present invention the antigenic determinant derived from ABC-T, where present, is between 30 to 50 kDa, and ideally around 40 kDa; the antigenic determinant derived from PavA, where present, is between 55 to 70 kDa, and ideally around 63 kDa; and the antigenic determinant derived from ZmpB, where present, is between 125 to 155 kDa, and ideally around 148 kDa. These sequences encompass the surface exposed fragments of the proteins deemed to comprise immune epitopes in flanking regions.

In an embodiment of the immunogenic composition of the present invention comprising an antigenic determinant derived from ABC-T, or a genetic construct encoding an antigenic determinant derived from ABC-T, the antigenic determinant comprises or consists of the protein sequence SEQ ID NO:1, or an immunologically effective fragment thereof, or an immunologically effective analog of the sequence or fragment thereof.

In an embodiment of the immunogenic composition of the present invention comprising an antigenic determinant derived from PavA, or a genetic construct encoding an antigenic determinant derived from PavA, the antigenic determinant comprises or consists of the protein sequence SEQ ID NO:2, or an immunologically effective fragment thereof or an immunologically effective analog of the sequence or fragment thereof.

In an embodiment of the immunogenic composition of the present invention comprising an antigenic determinant derived from ZmpB, or a genetic construct encoding an antigenic determinant derived from ZmpB, the antigenic determinant comprises or consists of the protein sequence SEQ ID NO:3, or an immunologically effective fragment thereof or an immunologically effective analog of the sequence or fragment thereof.

Preferably, the immunogenic composition of the present invention further includes an immunostimulatory agent, more preferably the immunostimulatory agent is an adjuvant as hereinafter described.

Preferably, the composition may include a further set of one or more antigenic determinants from Streptococcus pneumoniae bacterium that may be encapsulated or conjugated selected from the group comprising pneumococcal surface protein A (PspA), pneumococcal choline-binding protein A (PcpA), secreted 45-KDa protein Usp45-hydrolase (PcsB), serine/threonine protein kinase (StkP), peptide permease enzyme, manganese ABC transporter (PsaA), pneumolysin D (PlyD), pneumolysin toxoid (dPly), choline-binding protein A (CbpA) and D (CbpD), histidine triad protein D (PhtD), histidine triad protein E (PhtE), histidine triad protein A (PhtA), histidine triad protein B (PhtB); pneumococcal iron uptake protein A (PiuA), protein cell wall separation (PcsB), pneumococcal serine-threonine kinase (StkP) and analogs, plasmin and fibronectin-binding protein A (PfbB), SP0148, SP1912 and SP2108, SP0785, SP1500, SP2216.

According to a further aspect of the invention, there is provided the immunogenic composition described herein in the form of a kit, e.g. sealed in a suitable container which protects its contents from the external environment. Such a kit may include instructions for use.

Preferably, the immunogenic composition is packaged in a hermetically sealed container such as an ampoule or sachets indicating the quantity of composition. In one embodiment, the composition is supplied as a liquid, suspension, tablet or spray. In another embodiment, the composition is supplied as a dry sterilized lyophilized powder or water-free concentrate in a hermetically sealed container, wherein the composition can be reconstituted, for example, with water or saline, to obtain an appropriate concentration for administration to a subject.

When the vaccine of the present invention is systemically administered, for example, by subcutaneous or intramuscular injection, a needle and syringe, or a needle-less administration device, for example and without limitation, an inhalation or intranasal delivery can be used. The vaccine formulation can be enclosed in ampoules, disposable syringes or multiple dose vials made of glass or plastic.

The immunogenic compositions and vaccines of the present invention will generally be provided as a single composition, i.e. a single composition comprising all of the required components; however, it will be appreciated that the present invention is also directed to compositions and vaccines that are provided as multiple compositions, for example a first composition comprising one or more antigenic determinants and/or genetic constructs encoding one or more antigenic determinants, and a second composition comprising a different one of the specified antigenic determinants and/or genetic constructs encoding a different one of the specified antigenic determinants; or a first composition comprising one or more of the specified antigenic determinants and/or one or more genetic constructs encoding one or more antigenic determinants of the specified antigenic determinants, and a second composition comprising one or more adjuvants, either with or without further ones of the antigenic determinants and/or genetic constructs encoding further ones of the antigenic determinants. In this case the multiple composition may be administered simultaneously or sequentially. The present invention therefore further provides a method of treating or preventing a Streptococcus pneumoniae bacterium infection in an individual or immunising against a Streptococcus pneumoniae bacterium infection in an individual comprising administering a sufficient amount of at least one antigenic determinant derived from a protein selected from ABC-T, PavA and ZmpB of a Streptococcus pneumoniae bacterium and simultaneously or subsequently administering a sufficient amount of least one antigenic determinant derived from a protein selected from a different one of ABC-T, PavA and ZmpB of a Streptococcus pneumoniae bacterium.

The present invention further provides an immunological composition according to the present invention or a vaccine according to the present invention for use in the treatment or prevention of a Streptococcus pneumoniae bacterium infection in an individual and/or for immunising against a Streptococcus pneumoniae bacterium infection in an individual and/or diagnosing or screening for a Streptococcus pneumoniae bacterium infection in an individual.

The present invention further provides a kit comprising an immunogenic composition according to the present invention or a vaccine according to the present invention, and means to administer the immunogenic composition or vaccine to an individual. Optionally, the kits of the present invention may comprise additional components selected from one or more of carriers and means to resuspend the lyophilized composition or vaccine.

It will be appreciated that preferred features ascribed to one aspect of the invention applies mutatis mutandis to each and every aspect of the invention.

### BRIEF DESCRIPTION OF THE DRAWINGS

Embodiments of the invention are further described hereinafter with reference to the accompanying drawings, in which:

FIG. 1 shows SEQ ID NO:1, a truncated segment of the pneumococcal ATP-binding cassette transporter permease (ABC-T) protein sequence. Gene ID: 13695552, Gene symbol: HMPREF1038_02239, protein WP_000834690.1.

FIG. 2 shows SEQ ID NO:2, the pneumococcal adherence and virulence factor A (PavA) protein sequence.

FIG. 3 shows SEQ ID NO:3, a truncated segment of the pneumococcal zinc metalloprotease B (ZmpB) protein sequence.

FIG. 4 shows the immunobinding activity of human sera collected from children: n=13, Community Acquired Patients (CAP): n=5, Elderly patients: n=3, Healthy volunteers: n=5, and adults living with HIV: n=5. Mouse serum collected from naive wild-type mice was used as a negative control (Control serum) to show the absence of binding. Plates were coated with either PavA alone, ABC T alone, ZmpB alone or all 3 proteins combined (PAZ).

FIG. 5 shows validation of the role of the three pneumococcal antigens ABC-T, PavA and ZmpB in the invasion of the central nervous system. Viable counts collected in the brain tissues of a meningitis mouse model were determined at various time points (days) post-infection.

FIG. 6 shows clinical scores in mice immunised with a combination of three antigenic determinants derived from ABC-T, PavA and ZmpB, either alone or in combination with Alum or CpG-chitosan, FIG. 6A in neonates and FIG. 6B in adults. Abbreviations: Ag: mixture of ABC-T, PavA and ZmpB antigens; PCV-13: Pneumococcal Conjugate Vaccine (PCV-13/Prevenar-13).

FIG. 7A shows the results of antibody dose-response immunisation studies, FIG. 7B shows the results of cell-mediated immune response upon vaccination with vaccine formulations, FIG. 7C (antibody-mediated immunity) and FIG. 7D (cell-mediated immunity) show that the results of vaccination with vaccine formulations provide enhanced disease protection compared to conventional Alum adjuvant alone.

FIG. 8 shows the effective efficacy of vaccine and adjuvant formulations versus commercially available Prevenar-13® (PCV-13) in models of pneumococcal sepsis and pneumonia in adult and neonate mice. Kaplan-Meier survival curves are shown upon challenge with pneumococci in adult (FIG. 8A) and neonate (FIG. 8B) mice. Bacterial viable counts in the lung tissues and blood of neonate mice are shown in FIGS. 8C and 8D, respectively. Abbreviations: Ag: Antigen (combination of three antigenic determinants derived from ABC-T, PavA and ZmpB): PrPV: a composition of the present invention as described herein.

FIG. 9 shows that vaccine formulations of the present invention provide better immune protective efficiency against non-PCV13 responsive serotypes (i.e. serotypes 8, 11A and 33F) as compared to PCV-13, as percentage survival (FIG. 9A) and clinical pain scores (FIG. 9B).

FIG. 10 shows that vaccine formulations of the present invention activate/stimulate CD4+ T-cell responses ex vivo human peripheral blood mononuclear cells (PBMCs).

FIG. 11 shows that a vaccine formulation of the present invention activates plasma B cells in human PBMCs.

FIG. 12 shows the results from passive transfer experiments: sera from mice immunised with vaccine formulations of the present invention confers passive immunity in naïve mice, as demonstrated by mouse survival times (FIG. 12A), and colony forming units (CFU) (FIG. 12B) in blood and lung.

FIG. 13 shows the results from passive transfer experiments of human PBMC: concentrated supernatant from human PBMC cultures stimulated with vaccine formulations of the present invention confers passive immunity in naïve mice, as demonstrated by mouse survival times (FIG. 13A), and colony forming units (CFU) (FIG. 13B) in blood and lung.

FIG. 14 shows the results of an opsonophagocytosis killing assay from mice: immunisation with a vaccine formulation of the present invention (PrPv) promotes in vitro opsonization of pneumococci and outcompetes Prevenar-13 (PCV-13).

FIG. 15 show that vaccination with a mixed protein formulation of the present invention presents enhanced immunogenicity compared to single protein formulations adjuvanted with CpG Chitosan, FIG. 15A shows antibody dose-responses for antigen-specific IgG1 and IgG2a, FIG. 15B shows cell-mediated immune response upon vaccination.

FIG. 16 shows the vaccination with a mixed protein formulation of the present inventions provides enhanced disease protection as compared to single proteins adjuvanted with CpG Chitosan, FIG. 16A shows survival curves for adult mice and FIG. 16B colony forming units (CFU) in blood and lung.

FIG. 17 shows that the protein combinations (ABC-T+PavA+ZmpB) and (ABC-T+ZmpB) provide 100% protection (n=5 mice per group) and that the protein combinations (PavA+ABC-T) and (PavA+ZmpB) provide 60% protection, while 80% of the mice who received only vehicle solution succumbed to the infection within 46 hours of the administration (17A). Viable counts (CFUs) determined in blood (17B) and lung tissues (17C) are also shown. All of the formulations also included CpG-Chitosan.

FIG. 18 shows the results from nasal colonisation with Streptococcus pneumoniae post immunisation (a first prime and second booster) of antigens only (green), CpG-Chitosan only (blue) and antigens mixed with CpG-Chitosan (red) over 0, 1, 3, 9 and 15 days post immunisation, shown in panels A, B, C, D and E, respectively. Abbreviations: CFU: colony forming units; NP: Nasopharynx; pi: post-infection.

FIG. 19 shows the results from ex vivo immune cell proliferation (CD3+CD4+ cells collected from mesenteric lymph nodes) obtained in mice immunised with a mixed protein formulation of the present invention and subsequently colonised with S. pneumoniae. Staining panel shown: CD4-FITC; CD3-APC; Ki67—PEcy7.

FIG. 20 shows the induction of IL-17A positive memory T cells (CD62L+CD44+) in the lung tissues of mice immunised with vehicle, CpG-c
Chitosan alone, or a composition of the present invention (PrPV) i.e. ABC-T+PavA+ZmpB+CpG-Chitosan. Representative flow cytometry plots are shown in FIG. 20A, and group comparisons are shown in FIG. 20B. Abbreviation: MFI, Mean Fluorescence Intensity.

FIG. 21 shows the protective efficacy of immunising mice with a vaccine antigen formulation of the present invention including a beta-glucan-based adjuvant, distinct from CpG-Chitosan.

FIG. 22 shows SEQ ID NO:4, the mRNA sequence of the truncated segment of the pneumococcal ATP-binding cassette transporter permease (ABC-T) matching the amino acid sequence presented in FIG. 1 (SEQ ID NO:1).

FIG. 23 shows SEQ ID NO:5, the mRNA sequence of the pneumococcal adherence and virulence factor A (PavA) matching the amino acid sequence presented in FIG. 2 (SEQ ID NO:2).

FIG. 24 shows SEQ ID NO:3, the mRNA sequence of the truncated segment of the pneumococcal zinc metalloprotease B (ZmpB) matching the amino sequence presented in FIG. 3 (SEQ ID NO:3).

### DETAILED DESCRIPTION

The term “immunogenic composition” refers to any composition that has the ability to provoke an immune response in the body of a human or other animal, this could include, for example, production of antibodies, a T-cell and/or a B-cell mediated immune response, or any other such response to the introduction of a vaccine to the body which grants protection in the future from a disease or infection.

Reference herein to a “vaccine” refers to a biological preparation that is suitable to be put into the body of a person or animal and to provoke an immune response, as defined above.

As used herein the terms “treating” and “treatment” refer to any and all uses which remedy a condition or symptoms, prevent the establishment of a condition or disease, or otherwise prevent, hinder, retard, ameliorate or reverse the progression of a condition or disease or other undesirable symptoms in any way whatsoever.

The term “vaccine protein antigen” as used herein, refers to an antigen, regardless whether it is a protein-containing cell fragment, a purified protein, a synthetic peptide or whether in its nature it consists of amino acids only or of amino acids in combination with other biological molecules, such as carbohydrates or lipids, derived from an infectious organism against which the vaccine is intended to protect. The purpose of including the vaccine protein antigen is to induce specific and protective immunity towards the infection caused by the organism that the vaccine protein was derived from.

Reference herein to an “antigenic determinant” refers to a portion of a protein antigen to which antibodies, B-cells or T-cells may be directed, it is synonymous with an “epitope”.

Reference herein to an antigenic determinant “derived from” a particular protein refers to a peptide corresponding to all or part of the particular protein, or being an analog of all or part of protein, and having an antigenic effect similar to that of the protein. For example, an antigenic determinant derived from the protein ABC-T of a Streptococcus pneumoniae bacterium may comprise or consist of the entire sequence of the ABC-T protein, or it may comprise or consist of any segment of the protein, so long as it exhibits at least one antigenic effect exhibited by the ABC-T protein. Alternatively, the antigenic determinant derived from the protein ABC-T of a Streptococcus pneumoniae bacterium may comprise or consist of an analog of the entire sequence of the ABC-T protein, or a segment of an analog of the protein, so long as it exhibits at least one antigenic effect exhibited by the ABC-T protein. The same applies to antigenic determinants derived from the proteins PavA and ZmpB of a Streptococcus pneumoniae bacterium. Examples of suitable antigenic determinants derived from the proteins ABC-T, PavA and ZmpB of a Streptococcus pneumoniae bacterium comprise or consist respectively of the protein sequences SEQ ID NO:1, SEQ ID NO:2 and SEQ ID NO 3, or immunologically effective fragments thereof or immunologically effective analogs of the sequences or fragments thereof. In particular embodiments of the present invention, the antigenic determinant derived from ABC-T is from 30 to 50 kDa, and/or the antigenic determinant derived from PavA is from 55 to 70 kDa; and/or the antigenic determinant derived from ZmpB is from 125 to 155 kDa.

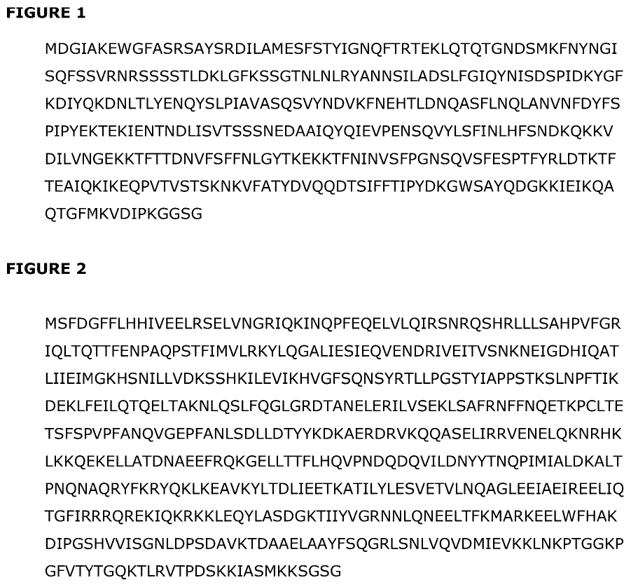

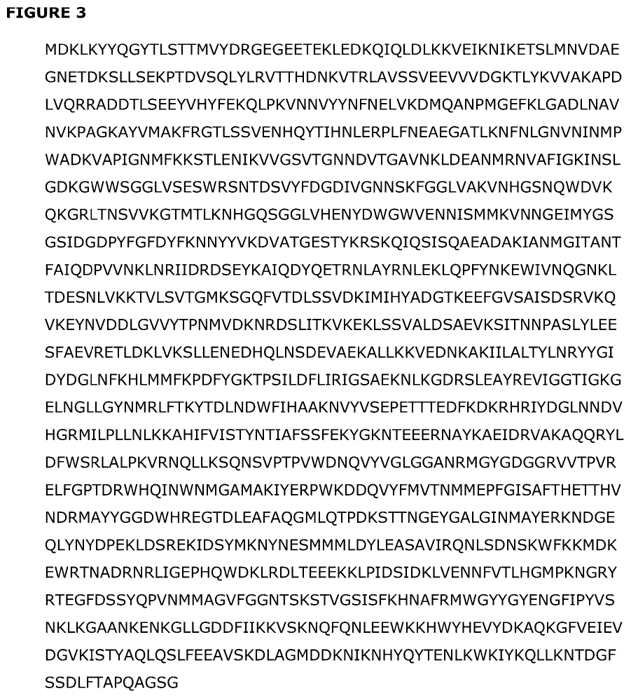

The proteins shown in FIGS. 1, 2 and 3/SEQ ID NOs: 1, 2 and 3 may include additional amino acids that are not part of the native sequence but are included in order to assist in recombinant production and/or purification of the protein, as long as these do not impact on the immunological properties of the antigen. Examples of suitable additional amino acids include a poly-histidine tag, such as (HHHHHH). In certain embodiments of the present invention, the truncated segment of ABC-T shown in FIG. 1 and/or the sequence of PavA shown in FIG. 2 and/or the truncated segment of ZmpB shown in FIG. 3 may include the terminal poly-histidine tag HHHHHH. When noting or calculating percent identity/homology the poly-histidine tag may not be part of the calculation.

Reference herein to an “analog” of a particular protein refers to a protein having sufficient identity/homology (the terms are used interchangeably herein) to the protein to exhibit at least one antigenic effect exhibited by the protein.

To determine the percent identity/homology of two amino acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 75%, 80%, 82%, 84%, 85%, 86%, 87%, 88%, 89%, 90%, 91%, 92%, 93%, 94%, 95%, 96%, 97%, 98%, 99%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap.

In an embodiment, an antigenic determinant which is an analog of ABC-T, PavA and ZmpB of a Streptococcus pneumoniae bacterium as used in the present invention has an amino acid sequence sufficiently or substantially identical to the amino acid sequences SEQ ID NOS: 1, 2 or 3 or a fragment thereof. The terms “sufficiently identical” or “substantially identical” are used herein to refer to a first amino acid sequence that contains a sufficient or minimum number of identical or equivalent (e.g. with a similar side chain) amino acid residues to a second amino acid sequence such that the first and second amino acid sequences have a common structural domain or common functional activity. For example, amino acid sequences that contain a common structural domain having at least about 60%, or 65% identity, likely 75% identity, more likely 85%, 90%, 91%, 92%, 93%, 94%, 95%, 96%, 97%, 98% or 99% identity are defined herein as sufficiently or substantially identical.

As used herein, an “immunologically effective analog or portion” of a protein includes a fragment of the protein that participates in an interaction eliciting the immunological response. Typically, immunologically effective analogs or portions of a protein comprise a domain or motif with at least one activity of the protein, e.g., the immunologically effective analog or portion may retain one or more immunogenic portions. A polypeptide has S. pneumoniae immunological effectiveness as defined herein, if it has one, two and preferably more of the following properties: (1) if when expressed in the course of an S. pneumoniae infection, it can promote, or mediate the attachment of S. pneumoniae to a cell; (2) it has an enzymatic activity, structural or regulatory function characteristic of an S. pneumoniae protein; (3) or the gene which encodes it can rescue a lethal mutation in an S. pneumoniae gene; (4) it contributes to the immune evasion properties of the bacterium. A polypeptide has immunological effectiveness if it is an antagonist, agonist, or super-agonist of a polypeptide having one of the above-listed properties.

An immunologically effective “fragment” or “analog” is one having an in vivo or in vitro activity which is characteristic of the S. pneumoniae polypeptides of the invention, particularly the proteins have the sequences of SEQ ID NOS: 1 to 3, or of other naturally occurring S. pneumoniae polypeptides, e.g., one or more of the biological activities described herein. Especially preferred are fragments which exist in vivo, e.g., fragments which arise from post-transcriptional processing or which arise from translation of alternatively spliced RNA. Fragments include those expressed in native or endogenous cells as well as those made in expression systems, e.g., in CHO cells. Because peptides such as S. pneumoniae polypeptides often exhibit a range of physiological properties and because such properties may be attributable to different portions of the molecule, a useful S. pneumoniae fragment or S. pneumoniae analog is one which exhibits a biological activity in any biological assay for S. pneumoniae activity.

Reference herein to a “genetic construct encoding an antigenic determinant” refers to any nucleic acid structure suitable for expressing the antigenic determinant when administered to a recipient, including DNA constructs (for example, a DNA plasmid), RNA constructs (for example, messenger RNA), bacterial-based constructs and viral-based constructs (such as inactivated or live-attenuated virus constructs). The genetic construct may encode antigenic determinants corresponding to the full sequence of a native protein, or they may encode immunologically effective fragments thereof or immunologically effective analogs of the native sequence or fragments thereof.

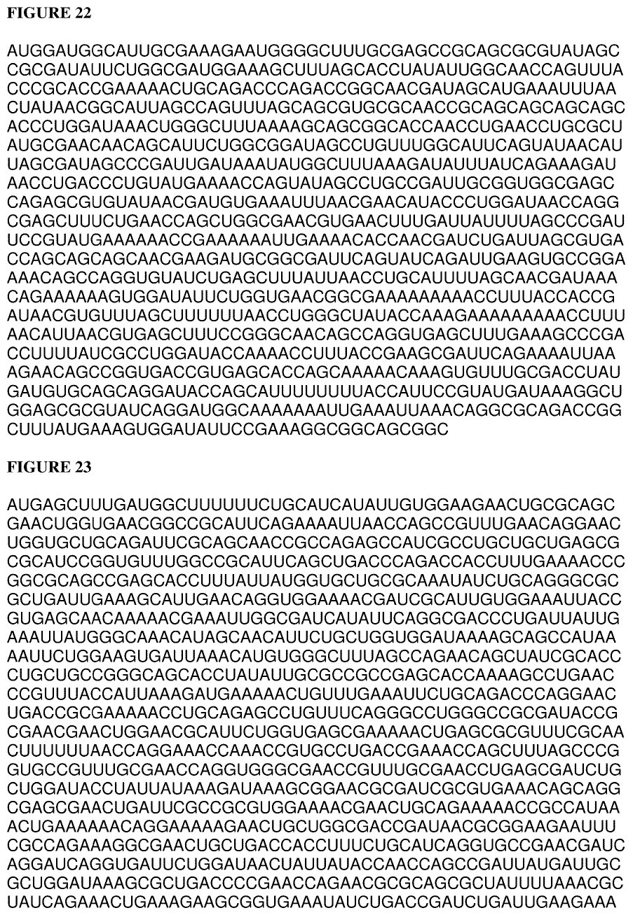

In embodiments of the present invention comprising a genetic construct encoding a protein comprising a fragment of ABC-T corresponding to the sequence of SEQ ID NO:1, the genetic construct may comprise a nucleic acid sequence (DNA or RNA) corresponding to SEQ ID NO:4 (i.e. the RNA sequence shown in FIG. 22, although the equivalent DNA sequence is also an embodiment of the present invention), or an analog thereof.

In embodiments of the present invention comprising a genetic construct encoding a protein comprising the protein PavA corresponding to the sequence of SEQ ID NO:2, the genetic construct may comprise a nucleic acid sequence (DNA or RNA) corresponding to SEQ ID NO:5 (i.e. the RNA sequence shown in FIG. 23, although the equivalent DNA sequence is also an embodiment of the present invention), or an analog thereof.

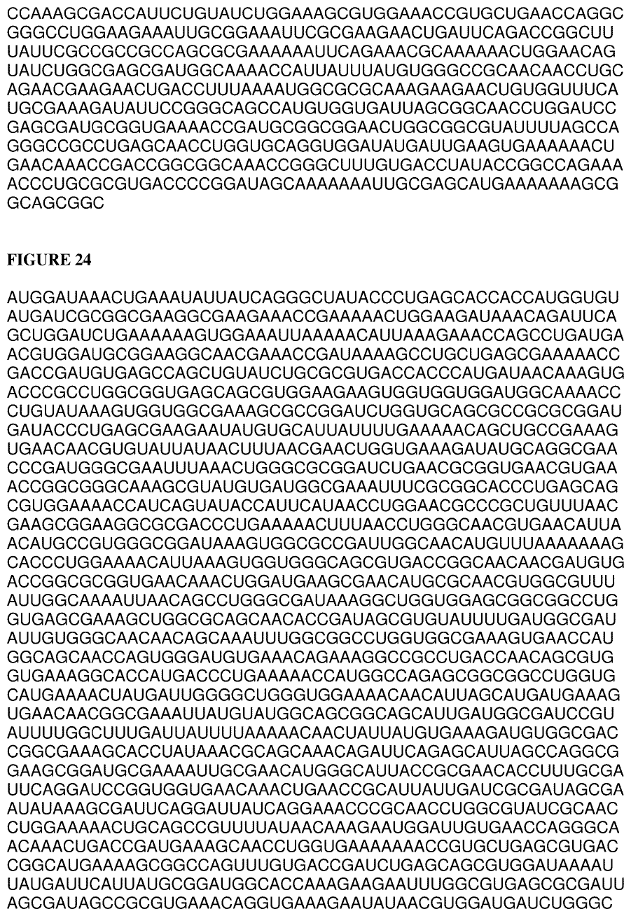

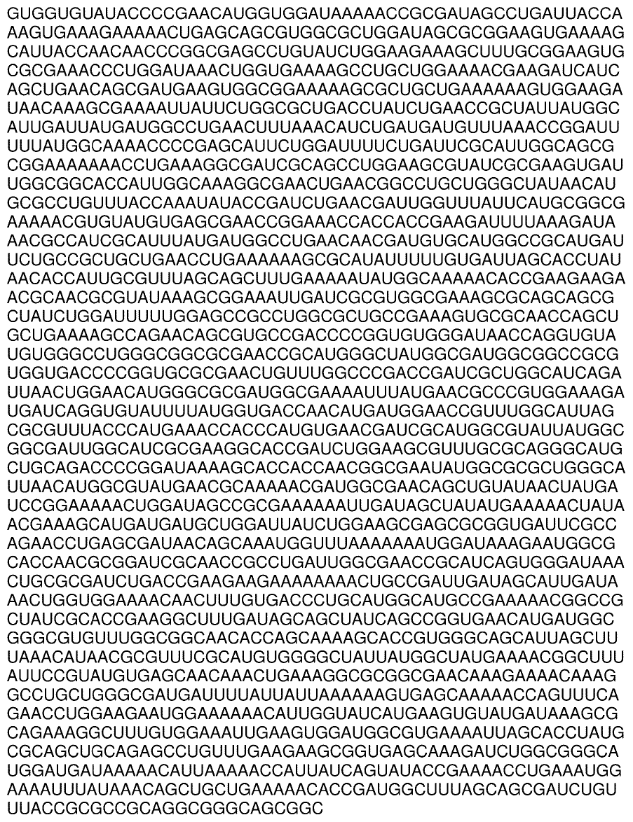

In embodiments of the present invention comprising a genetic construct encoding a protein comprising a fragment of ZmpB corresponding to the sequence of SEQ ID NO:3, the genetic construct may comprise a nucleic acid sequence (DNA or RNA) corresponding to SEQ ID NO:6 (i.e. the RNA sequence shown in FIG. 24, although the equivalent DNA sequence is also an embodiment of the present invention), or an analog thereof.

Reference herein to “immunostimulation” refers to immunostimulants and immuno-stimulators that stimulate the immune system by inducing activation or increasing activity of any of its components.

Reference herein to an “adjuvant” is intended to include a pharmacological or immunological agent that modifies the effect of other agents, in this instance the vaccine compositions of the present invention. The adjuvants used in the present invention act to boost or enhance the magnitude and/or durability of an immune response of the vaccine composition which may also lead to reducing the amount of antigens required to induce protective and long lasting immunity.

Proteins, protein fragments and genetic constructs used in vaccine compositions are often conjugated or mixed with immunostimulatory or immune-potentiating substances/adjuvants. The incorporation of adjuvants into vaccine formulations is aimed at enhancing, accelerating and prolonging the specific immune response to vaccine antigens. Advantages of adjuvants include the enhancement of the immunogenicity of weaker antigens, the reduction of the antigen amount needed for a successful immunisation, the reduction of the frequency of booster immunisations to achieve an adequate level of protective immunity. Selectively, adjuvants can also be employed to optimise a desired immune response, e.g. with respect to immunoglobulin classes and induction of cytotoxic or helper T lymphocyte responses.

In an embodiment the immunogenic compositions of the present invention may comprise one or more materials selected from materials having a stimulatory effect on Toll-Like Receptors (TLR), cytosolic pattern recognition receptors (PRRs) such as nucleotide-binding oligomerization domain (NOD)-like receptors (NLRs), C-type lectin receptors (CLRs), nucleic acid-sensing receptors such as retinoic acid-inducible gene I (RIG-I), or nutrient sensors such as mTOR and GCN2; materials acting on at least one of the following pathways: MyD88/TLR-signalling pathways, cGAS-stimulator of interferon genes (STING) pathway, NF-κB pathway, any stress, cell death and tissue damage pathways (such as necrosis/necroptosis, apoptosis, autophagy) resulting in release of damage-associated molecular patterns (DAMPs) such as nucleic acids, uric acid, ATP and proteins that activate the innate immune system, any signalling pathways resulting in immune cell recruitment or epigenetic changes that maintain the innate immune system at an alarming state for extended periods i.e. a memory-like state; materials having the property to induce inflammasome activation ensued by cell differentiation to a CD4+ Th2 (interleukin-4), Th1 (Interferon-gamma)- or Th17(IL17A)-mediated immunity; immune potentiators; polysaccharide-based materials acting on the IL-1β, CLRs and TNF-α signalling pathways); delivery systems and mucosal adjuvants.

Additionally or alternatively, the immunogenic compositions of the present invention may comprise one or more materials selected from bacterial proteins and/or polysaccharide materials such as capsules and analogs thereof, toxin/toxoid and analogs thereof, TLR ligands and analogues thereof, agonists such as Pam3CSK4, Pam2CSK4, MPLA (LPS derivative), CpG (short single-stranded synthetic DNA molecules that contain an unmethylated cytosine triphosphate deoxynucleotide (“C”) followed by a guanine triphosphate deoxynucleotide (“G”), the “p” referring to the phosphodiester link between consecutive nucleotides, although some ODN have a modified phosphorothioate (PS) backbone instead), PolyI:C, CpG motifs; microparticles and nanoparticles such as chitosan or beta-glucans, flagellin, poly(lactic-co-glycolic acid) or PLGA-poly-1-lysine/poly-gamma-glutamic acid (PLGA-PLL/gammaPGA), or made of lipid-based backbone co-formulated with any other immunostimulatory agents such as monophosphoryl lipid A (MPLA), polyethylene glycol (PEG), oligomannose, Poly(I:C), extracellular vesicles (EV), such as outer membrane vesicles (OVM); oil-based encapsulation or emulsion-based systems, including water-in-oil, oil-in-water and thermo reversible oil-in-water emulsions such as Montanide ISA-51, Montanide ISA-720 and analogs thereof, alum or aluminium salt adjuvants, liposomes, virosomes, archeosomes, outer-membrane vesicles, niosomes, saponins, and immunostimulating complexes (ISCOMs), polymeric particles, cytokines including proinflammatory cytokines such as IFN-γ, IL-1, IL-2, IL-4, IL-12, IL-17A/F, GM-CSF and MPI, virus-like particles (VLPs), bacterial components such as detoxified variant of LPS such as Monophosphoryl lipid A (MPL), muramyl dipeptide (MDP lipophilic and hydrophobic), QS21, PLGA, CT, liposome-based cationic adjuvant formulation e.g. CAF 01-09 (composed of Span80, polyoxyethylene cetyl-stearylether, mannitol, squalene) and analogs composed of cholesterol, phosphatidylcholine and phosphatidylserine, or alpha-Gal ceramide, NOD-like receptor protein 3 (NLRP3) inflammasome, Apoptosis-associated speck-like protein containing a CARD (ASC), NLR-family CARD domain-containing protein 4 (NLRC4)inflammasome, Absent in melanoma 2(AIM2)inflammasome, dsRNA: Poly(I:C), Poly-IC:LC, Monophosphoryl lipid A (MPL), LPS, Flagellin, Imidazoquinolines: imiquimod (R837), resiquimod (848), CpG oligodeoxynucleotides (ODN), Muramyl dipeptide (MDP), Saponins (QS-21), bacterial molecules or derivatives including polysaccharide capsules and analogs thereof, toxin/toxoid and analogs thereof, unmethylated DNA (CpGs) and analogs thereof, cytokines such as IL-2, IL-12, TNF-α, and granulocyte-macrophage colony-stimulating factor (GM-CSF), chemokines such as RANTES (regulated on activation, normal T cell expressed and secreted), macrophage inflammatory protein (MIP)-1α, costimulatory or adhesion molecules such as CD80, lymphocyte function-associated antigen-3 and polyarginine tails, polysaccharide-based materials with properties similar to those presented by chitosan, such as CpG-delta Inulin, cochleates, virus-like particles (VLP), microparticulates such as virosomes, PLA (polylactic acid), PLG (poly[lactide-coglycolide]), Cholera toxin (CT) derivatives, and Heat-labile enterotoxin (LTK3 and LTR72).

In particular embodiments, the immunological compositions of the present invention may comprise one or more materials selected from TLR agonists, agents capable of inducing a CD4+ T-cell mediated immune response (particularly with a Th1- and/or Th17-differentiated profile), oil-in-water emulsions, water-in-oil emulsions, agents acting on the MyD88/TLR-signalling pathway, agents acting on the cGAS-stimulator of interferon genes (STING) pathway and particulate polysaccharide materials. In particularly preferred embodiments of the immunological compositions of the invention, the adjuvant comprises a combination of a TLR agonist and an agent capable of acting on the MyD88/TLR-signalling pathway or the cGAS-stimulator of interferon genes (STING) pathway; for example, a combination of CpG with chitosan and/or beta-glucans.

Lyophilization of vaccines is well known in the art. Typically, the liquid vaccine is freeze-dried in the presence of a clot-inhibiting agent, such as a saccharide, e.g. sucrose or lactose (which is present at an initial concentration of 10 to 200 mg/ml). Lyophilization usually takes several steps, for example, the cycle begins at −69° C., gradually adjusts the temperature to −24° C. over 3 hours, then maintains that temperature for 18 hours, then gradually adjusts to −16° C. and then to 1° C. hours, then this temperature is maintained for 6 hours and then adjusted to +34° C. over 3 hours and finally maintained for 9 hours.

Lyophilization of the formulation results in a more stable formulation (e.g., degradation of polysaccharide antigens is prevented. This process is also surprisingly responsible for higher antibody titres against pneumococcal polysaccharides. This has been found especially for PS 6B conjugates and a 3DMPL adjuvant (preferably no aluminium-based adjuvant) and a pneumococcal protein selected from the group consisting of: PhtA, PhtB, PhtD, PhtE, SpsA, LytB, LytC, LytA, Spl25, Spi01, Spl28, Spl30 and Spi33.

In some embodiments of the invention the immunogenic composition or vaccine may be lyophilized.

In another aspect, the invention encompasses: a vector including a nucleic acid which encodes an S. pneumoniae polypeptide or an S. pneumoniae polypeptide variant as described herein; a host cell transfected with the vector; and a method of producing a recombinant S. pneumoniae polypeptide or S. pneumoniae polypeptide variant; including culturing the cell, e.g., in a cell culture medium, and isolating an S. pneumoniae polypeptide or an S. pneumoniae polypeptide variant, e.g., from the cell or from the cell culture medium.

Methods are also provided for producing antibodies in a host animal. The methods of the invention comprise immunizing an animal with at least one S. pneumoniae-derived immunogenic component, wherein the immunogenic component comprises the immunogenic composition or vaccine of the present invention or sequence-conservative or function-conservative variants thereof, or polypeptides that are contained within any ORFs, including complete protein-coding sequences, of which any of SEQ ID NO:1, SEQ ID NO:2 or SEQ ID NO:3 forms a part; or polypeptide sequences contained within any of SEQ ID NO:1, SEQ ID NO:2 or SEQ ID NO:3 or polypeptides of which any of SEQ ID NO:1, SEQ ID NO:2 or SEQ ID NO:3 forms a part. Host animals include any warm blooded animal, including without limitation mammals and birds. Such antibodies have utility as reagents for immunoassays to evaluate the abundance and distribution of S. pneumoniae-specific antigens.

Delivery systems that have been studied to achieve the design of more efficient peptide-vaccines include, nanoscale size (<1000 nm) materials such as virus-like particles (VLPs), outer-membrane vesicles (OMVs), liposomes, immune stimulating complexes (ISCOMs), polymeric, and non-degradable nanospheres have received attention as potential delivery vehicles for vaccine antigens which can both stabilize vaccine antigens and act as adjuvants. Besides, they offer the ability to design vaccines containing multiple protein antigenic fragments that specifically target immune cells, leading to more effective uptake by antigen-presenting cells.

Alternative delivery systems may include biodegradable materials e.g. natural polymeric compounds such as starch, alginates and cellulose, biosynthetic materials such as Poly beta-hydroxybutyrate (PHB), and co-polymers such as Polylactic acid (PLA), polyurethane, Poly Lactic-co-Glycolic Acid (PLGA) and Polymethyl methacrylate resin (PMMA), have received a lot of attention in vaccine research because of their biocompatibility, biodegradability and often low toxicity, which can protect antigens from degradation, increase antigen stability and provide slow release; resulting in enhanced overall immunogenicity. These are also prospective adjuvants/delivery systems for the novel pneumococcal vaccines of the present invention.

Further delivery systems include, for example and without limitation: probiotics, from for example the Lactobacilli and Bifidobacteria species; viral vector systems such as modified Vaccinia virus Ankara (MVA) and other various viruses, including poxviruses and adenoviruses and; bacteriophages such as phage T4.

It will be appreciated that the vaccine of the present invention may be used in conjunction with other existing vaccines/conjugated vaccines. The vaccine of the present invention may be used as a carrier protein to improve currently available formulations, for example and without limitation Prevenar contains a carrier protein CRM197. Accordingly, CRM197 may be substituted by one or more or all of the proteins/polypeptides of the present invention.

A composition described herein may be administered alone or in combination with other treatments, either simultaneously or sequentially. Administration may be repeated at daily, twice-weekly, weekly or monthly intervals. The treatment schedule for an individual subject may be dependent on factors such as the route of administration and the severity of the condition being treated.

The present invention also encompasses combination vaccines that provide protection against various pathogens. Many pediatric vaccines are currently administered as combination vaccines to reduce the number of injections a child must receive. Thus, pediatric vaccines containing antigens from other pathogens may be formulated with the vaccines of the present invention. For example, the vaccines of the present invention may be formulated with (or administered alone, but at the same time) a well-known “trivalent” combination vaccine containing Diththeric Toxoid (DT), Tetanus Toxoid (TT), a Pertussis component [typically detoxified filamentous haemugglutinin (FHA) optionally with pertactin (PRN) and/or agglitomome, 1+2], for example with the marketed INFANRIXDTPa™ vaccine (SmithKline Beacham Biologicals) containing DT, TT, PT, FHA and PRN antigens, or with the pertussis component whole cell, such as the vaccine marketed by SmithKline Beecham Biologicals S.A. as Tritanrix™. The combination vaccine may also contain additional antigen, such as hepatitis B virus surface antigen (HBsAg), Polio virus antigens (for example, inactivated trivalent polio virus—IPV), Moraxella catarrhalis outer membrane proteins, non-typical Haemophilus influenzae proteins, outer membrane N. meningitidis B.

The composition may be administered in one or more doses which may be followed by one or more further ‘booster’ doses which are administered days, weeks or years later. For example, when administering the composition to children, a first dose may be given at 1 to 12 years of age and a booster dose at 16 years of age. For adolescents who receive the first dose at 13-15 years of age, a booster dose may be given at 16-18 years of age. For individuals who may not have received any pneumococcal vaccine in early childhood, a prime and/or booster dose of the immunogenic composition or vaccine of the invention may be administered or co-administered with seasonal vaccines such as the seasonal influenza/flu vaccine. The immunogenic composition or vaccine of the invention may also be administered as a prime or booster in adults aged over 65 which may have received one or 2 doses of PPSV-23 (Pneumovax) at a 1- or 2 year-interval. The injections may contain the same dose of active ingredient or may contain different doses. Preferably the dose will be administered by injection, although needle-free administration is also within the scope of the invention.

The immunogenic composition or vaccine may be administered to children, adolescents, or adults. ‘Children’ includes infants who are generally those up to 2 years of age. In some countries, infants will generally be administered two doses with a third ‘booster’ dose administered in the second year of life, but in other countries only 2 doses may be administered and in others, four doses may be used.

The specific dose level and frequency of dosage for any particular patient may be varied and will depend upon a variety of factors including the age, body weight, general health, sex, diet, mode of administration of the individual undergoing treatment. Typically, a suitable dosage may be determined by a physician. The composition may be administered as a dose from 0.00001 μg/Kg body weight to body weight to 5 mg/Kg body weight, preferably 0.0001 μg/Kg to 5 mg/Kg, preferably 0.001 g/Kg to 1 mg/Kg, preferably 0.01 g/Kg to 500 g/Kg, preferably 0.02 μg/Kg to 300 g/Kg body weight.

Currently, there are two types of licensed pneumococcal vaccines both targeting capsular polysaccharides: (a) the 23-valent pneumococcal polysaccharide-based vaccine (PPV-23, sold under the tradename Pneumovax) and; (b) the 7-, 10-, and 13-valent pneumococcal conjugate vaccines (PCV-7, -10, -13, sold under the tradename Prevenar). However, PPV-23 is poorly immunogenic in children under two years of age and generates neither an immune memory response nor herd immunity. Conjugation of a pneumococcal polysaccharide to a carrier protein turned polysaccharide-based vaccines from T-cell independent to T-cell dependent antigens, enhancing their immunogenicity. Conjugate vaccines evoked an efficient immune memory and induced relevant herd immunity.

Other suitable pneumococcal vaccines that could be used in conjunction with the vaccine of the present invention include, whole cell pneumococcal vaccine which is an inactivated cellular preparation of a non-encapsulated strain of S. pneumoniae, in which the autolysin (lytA) gene was deleted and the pneumolysin (ply) gene substituted for pdT. Additionally other suitable candidates include one or more selected from the group comprising pneumococcal surface protein A (PspA), pneumococcal choline-binding protein A (PcpA), secreted 45-KDa protein Usp45-hydrolase (PcsB), serine/threonine protein kinase (StkP), peptide permease enzyme, manganese ABC transporter (PsaA), pneumolysin D (PlyD), pneumolysin toxoid (dPly), choline-binding protein A (CbpA) and D (CbpD), histidine triad protein D (PhtD), histidine triad protein E (PhtE), histidine triad protein A (PhtA), histidine triad protein B (PhtB); pneumococcal iron uptake protein A (PiuA), protein cell wall separation (PcsB), pneumococcal serine-threonine kinase (StkP) and analogs, plasmin and fibronectin-binding protein A (PfbB), SP0148, SP1912 and SP2108, SP0785, SP1500, SP2216.

The vaccine compositions of the present invention can be administered by any conventional route, such as orally (for example tablet or capsule), nasally (for example a spray), inhalation, topical (for example a cream or lotion) and injection. The administration may be, for example, intravenous, intraperitoneal, intramuscular, intracavity, subcutaneous, or intradermally. The vaccine compositions of the invention are administered in effective amounts. An “effective amount” is that amount of a vaccine composition that alone or together with further doses, produces the desired response. In the case of preventing a pneumococcal disease the desired response is providing protection when challenged by an infective agent.

The compositions described herein may be formulated with a pharmaceutically acceptable carrier, excipient, buffer, stabilizer or diluent or other materials well known to those skilled in the art. Suitable pharmaceutically acceptable carriers, excipients or diluents are described, for example, in (Remington's Pharmaceutical Sciences, 18th edition, A. R. Gennaro, Ed., Mack Publishing Company [1990]; Pharmaceutical Formulation Development of Peptides and Proteins, S. Frokjaer and L. Hovgaard, Eds., Taylor & Francis [2000]; and Handbook of Pharmaceutical Excipients, 3rd edition, A. Kibbe, Ed., Pharmaceutical Press [2000]). The precise nature of the carrier or other material will depend on the route of administration.

The composition may be formulated in a form which is appropriate for the intended mode of administration. For example, as a powder, spray, tablet, solution, or suspension, optionally together with suitable carriers, excipients or diluents (or a combination thereof). For parental administration the composition may be in the form of a sterile aqueous solution and may optionally contain other substances, for example salts or buffers. Those of skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required including buffers such as phosphate, citrate and other organic acids; antioxidants, such as ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3′-pentanol; and m-cresol); low molecular weight polypeptides; proteins, such as serum albumin, gelatin or immunoglobulins; hydrophilic polymers, such as polyvinylpyrrolidone; amino acids, such as glycine, glutamine, asparagines, histidine, arginine, or lysine; monosaccharides, disaccharides and other carbohydrates including glucose, mannose or dextrins; chelating agents, such as EDTA; sugars, such as sucrose, mannitol, lactose, trehalose or sorbitol; salt-forming counter-ions, such as sodium; metal complexes (e.g. Zn-protein complexes); and/or non-ionic surfactants, such as TWEEN™, PLURONICS™ or polyethylene glycol (PEG).

A ‘therapeutically effective amount’ means a sufficient amount of a composition to show benefit to a subject, including, but not limited to, inducing/increasing an immune response against Streptococcus pneumoniae bacteria in a subject, and/or reducing the severity or duration of disease in a subject. The actual amount administered, and rate and time-course of administration, will depend on the nature and severity of what is being treated. Prescription of treatment, e.g. decisions on dosage etc, is within the responsibility of general practitioners and other medical doctors and may depend on the severity of the symptoms and/or progression of a disease being treated.

An immune response is induced or increased if these is a detectable difference in an immunological response indicator measured before and after administration of a particular composition. Immune response indicators include but are not limited to: antibody titer or specificity, as detected by an assay such as enzyme-linked immunoassay (ELISA), bactericidal assay, flow cytometry, immunoprecipitation, Ouchterlony immunodiffusion; binding detection assays of, for example, spot, Western blot or antigen arrays; cytotoxicity assays, ELISpot, ex vivo peripheral blood monocytic cell and lymphocyte stimulation assays, murine peritonitis and challenge models, etc.

In some embodiments, the therapeutically effective amount of vaccine formulation or composition described herein is an amount sufficient to generate antigen-specific antibodies (e.g., anti-Streptococcus pneumoniae bacterium). In some embodiments, the therapeutically effective amount is sufficient to provide seroprotection in a subject; i.e., to generate sufficient antigen-specific antibodies to prevent/protect from infection. In some embodiments, seroprotection is conferred on at least 60%, 70%, 80%, 90%, or at least 95% of vaccinated subjects. In some embodiments, an effective amount of the immunogenic compositions or vaccines of the present invention is sufficient to seroconvert a subject with at least 50% probability. In some embodiments, the therapeutically effective amount is sufficient to seroconvert a subject with at least 60%, 70%, 80%, 90% or at least 95% probability. Whether a subject has been seroconverted can be assessed by any method known in the art, such as obtaining a serum or peripheral blood sample from the subject and performing an assay to detect anti-Streptococcus pneumoniae antibodies or S. pneumoniae-induced lymphocyte responses. In some embodiments, a subject is seroconverted if a serum sample from the subject contains an amount of anti-Streptococcus pneumoniae antibodies or lymphocyte-mediated responses that surpasses a threshold or predetermined baseline.

In Vitro Investigations

Bacterial Isolates

Streptococcus pneumoniae serotype-2, lab-adapted strain D39 (NCTC 7466), was obtained from the National Collection of Type Culture (London, UK). All other pneumococcal strains used in the study, e.g. hypervirulent pneumococcal serotype 1, were clinical isolates obtained from biobank of isolates archived at the Liverpool Royal Hospital (RLBUHT) or at the Malawi Wellcome Trust Centre, Blantyre, Malawi. Pneumococcal identification was confirmed by optochin sensitivity test and serotypes were determined using the standard Quellung reaction with pneumococcal capsule-specific antisera (Statens Serum Institute, Copenhagen, Denmark). All isolates were stored in the lab at −80° C. on Protect™ cryobeads (Pro-Diagnotics Lab, Inc.) and subcultured on 5% horse blood agar plates incubated at 37° C. under anaerobic conditions for 18 hours. Multilocus sequence typing (MLST) was also carried out on all isolates.

Tissue samples for counting bacterial viable counts.

The viable counts of bacteria in blood and lungs were determined at the indicated intervals after pneumococcal infection. Tissue samples were ground using an IKA T10 homogeniser. Viable counts in lung cell suspension and in blood samples were determined by serial dilution in sterile PBS and plating on blood agar containing 5% (v/v) defibrinated horse blood (Oxoid) and 10 ug/ml gentamicin (sigma). Once dry, plates were incubated in 5% (v/v) CO2 at 37C overnight and bacterial colony numbers counted on the following day.

Mutagenesis of Streptococcus pneumoniae.

Generation of competent S. pneumoniae cells and subsequent transformation were performed using the complete transformation medium (C+Y media, pH=6.8) method (Lacks and Hotchkiss, 1960). Single deletion mutants were constructed by replacing the open reading frame (ORF) with the aphA3 cassette (conferring kanamycin resistance) and subsequent selection of kanamycin-resistant recombinants, on blood agar base medium supplemented with kanamycin. The recombination was verified by Sanger sequencing.

Recombinant protein production and adsorption onto CpG-Chitosan.

Escherichia Coli-optimised synthetic genes for each of the vaccine candidates were obtained from GeneArt (Thermo Fisher Scientific). These comprised a truncated segment of ABC-T corresponding to the sequence shown in FIG. 1 (SEQ ID NO: 1), the full sequence of PavA corresponding to the sequence shown in FIG. 2 (SEQ ID NO:2) and a truncated segment of ZmpB corresponding to the sequence shown in FIG. 3 (SEQ ID NO:3).

For each antigen, 6×His-tagged C-terminal recombinant pneumococcal proteins were cloned into E. Coli BL21 (DE3) cultured in Terrific Broth (Thermo Fisher Scientific). ABC-T is a large, multiple transmembrane protein: the sequence from Met435 to Gly790 (SEQ ID NO:1, approximately 40 kDa) was amplified and sub-cloned as a T7 promoter C-term 6×His tag recombinant entity. PavA was expressed in its full length i.e., from Met1 to Ser551 (SEQ ID NO:2, approximately 63KDa) as a T7 promoter C-term 6×His tag recombinant protein. The largest extracellular domain of ZmpB, i.e., Met140 to Ala1876 (SEQ ID NO:3, approximately 148 kDa) was amplified and sub-cloned as a T7 promoter C-term 6×His tag truncated protein. All 6×His-tagged proteins were recovered from IMAC Nickel (Bio-Rad laboratories) columns and passed four times through Pierce™ High-capacity endotoxin removal spin columns (Thermo Scientific Fisher), and residual endotoxin levels were confirmed to be less than 0.05 units/ml. The expressed proteins were then combined with a chitosan (CS) polymer (Tradename Protosan, Novomatrix) as a single protein or combined protein formulations (i.e. formulations comprising pairs of the proteins or all three proteins), i.e., 1-10 μg of antigen per vaccine formulation. Pneumococcal antigens adsorbed CS nanoparticles were formed upon stirring of sterile aqueous phosphate buffer saline solution containing 100 μg/ml of pneumococcal antigens (as single or combined formulation, 1-10 ug of antigen per vaccine formulation). The CS nanoparticles were then mixed with CpG (Unmethylated cytosine—phosphorothioate—guanine oligode oxynucleotides or CpG ODN 1826 (group 2), supplied by Integrated DNA Technologies Inc.

A mixture of the three antigens corresponding to SEQ ID NOS: 1, 2 and 3 in the absence of any adjuvants is an immunological composition of the invention, and is referred to herein as “Antigens”.

The above compositions comprising a mixture of two of the three antigens corresponding to SEQ ID NOS: 1, 2 and 3 absorbed on to chitosan microparticles in combination with CpG are immunological compositions of the present invention, and are referred to herein by reference to their respective antigenic determinants, i.e. “ABC-T+ZmpB”, “PavA+ZmpB” and “PavA+ABC-T”.

The above composition comprising a mixture of the three antigens corresponding to SEQ ID NOS: 1, 2 and 3 absorbed on to chitosan microparticles in combination with CpG is a preferred immunological composition of the present invention, and is referred to herein as “PrPV” (Protein-based Pneumococcal Vaccine).

ABC-T was found to be a large, multiple transmembrane domains, highly insoluble/toxic in E coli. The largest external domains were expressed as per design based on TMHMM server analysis. PavA, full length protein was expressed. ZmpB was determined to be a very large, multiple functional domains as identified by NCBI Domain Homology search. Trial expression of fragments of the protein were carried out to see which largest portion gave the best expression.

Opsonophagocytosis assay (OPKs).

The aim of this assay is to reproducibly measure the opsonization capacity of test serum samples against Streptococcus pneumoniae using a method adapted from the published standardized CDC method (Steiner et al Clinical and Diagnostic Laboratory Immunology 4: 415: 1997). This assay mimics in vitro conditions that are the primary mechanism in vivo to eliminate invading Streptococcus pneumoniae, followed by phagocytosis and then killing microorganisms. “Phagocytosis” is the process by which cells absorb material and enclose it in a vacuole (phagosome) in the cytoplasm. Pneumococci are killed when they are phagocytosed by healthy mammalian phagocytes. “Opsonization” is a process that facilitates phagocytosis by depositing opsonins, such as antibodies and complement, on an antigen.

Many opsonophagocytic assays have been described in the literature. The standardized CDC method has been tested in many laboratories (Steiner et al., ICAAC, Sep. 16-20, 2000, Toronto). This test was then modified in the SB, providing the basis for comparison for other laboratories, using reagents and controls that are generally available, and expressing results as serum titer (dilution) that facilitates killing 50% of viable pneumococci, a unit that is commonly used for this type of test. In addition, it has been shown that this modified test can generate results that correspond well with 4 other laboratories (Steiner et al., ICAAC, Sep. 16-20, 2000, Toronto).

OPKs were performed to test the ability of vaccinated mouse antiserum to opsonise viable pneumococci and mediate killing. A reference opsonophagocytic assay described elsewhere (Bangert et al, 2012 JID) was used with minor modifications. A reaction mixture consisting of heat-inactivated serum obtained from vaccinated vs. unvaccinated mice, differentiated HL-60 cells or freshly isolated human neutrophils, pneumococci, and baby rabbit complement were prepared. Control reactions lacking complement and/or antibody, effector cells or all components except pneumococci were run in parallel and incubated at 37° C. while shaking. Aliquots were removed prior to and after incubation, serially diluted in sterile PBS and samples plated on blood agar plates for CFU counts. A total of 5×10⁴ DMF-differentiated HL-60 cells were incubated with 5×10² opsonized S. pneumoniae and complement, for 45 min at 37° C./5% CO₂. Viable colony counts were performed after approximately 18 hours incubation period under anaerobic conditions at 37° C. IVIG at a final dilution of 1:16 was used as the source for pathogen-specific antibody for opsonization. Wells containing nonopsonized pneumococci and heat-inactivated complement were used as controls.

Ex Vivo Investigations

PBMC isolation and ex vivo stimulation assay

PBMCs were isolated from whole blood by Ficoll-Paque Plus (GE Healthcare Bio-Sciences). In brief, whole blood was collected into heparin tubes and diluted with an equal volume of DPBS in 50 ml Falcon tubes. The 30 ml volume of diluted blood was layered on top of the Ficoll, and centrifuged at 400 g for 30 min at room temperature. Mononuclear cells (MNCs) form a defined cell layer at the interface, between the plasma and Ficoll. The top layer (plasma) was carefully removed and then the MNC layer was collected into a fresh 50 ml Falcon tube. MNCs were washed by topping up with DPBS and centrifuged at 300 g for 10 min. Supernatant was discarded, and cell pellet was resuspended with complete RPMI1640 media supplemented with 10% FBS, 1% Penicillin/Streptomycin and 200 mM L-glutamine. Intracellular cytokine (IFN-gamma and IL-17A) assays were performed using PBMCs to examine their antigen-specific and T cell responses to the 3 pneumococcal antigens. Briefly, PBMCs (1×10⁶/well) were freshly cultured for 72 hours (37° C., 5% CO2) in RP10 (RPMI-1640 containing 10% FBS, 1 mM GlutaMAX-I supplement, 55 μM 2-mercaptoethanol, 50 U/mL penicillin, 50 μg/mL streptomycin, and 10 mM HEPES) in the presence of pneumococcal antigens adsorbed onto CpG-Chitosan. Brefeldin A (BFA; 5 μg/mL; Sigma-Aldrich) was included for the final 14 hours. PBMCs were then fixed, permeabilised, and labelled with cell-surface-marker-specific antibodies (anti-CD3 FITC, anti-CD4 PerCP Cy5.5, and anti-CD8 APC; all from BD Biosciences) and anti-IFN-PE and IL-17A-APC (Invitrogen Corporation) to detect intracellular cytokines. Proliferative responses were assessed using Ki67-labeling.

In Vivo Murine Investigations

Mouse nasopharyngeal carriage model.

S. pneumoniae were streaked onto blood agar and grown overnight at 37° C., 5% CO₂. S. pneumoniae were identified by presence of a zone of alpha haemolysis round each colony and a zone of inhibition round an optochin disc. A sweep of colonies was inoculated into brain heart infusion (BHI) broth and grown statically overnight at 37° C. The next day 750 μl of overnight growth was sub-cultured into BHI containing 20% (v/v) fetal calf serum (FCS) and grown statically for 4-6 hours until mid-log phase growth (OD500 0.8), at which point the broth was divided into 500 μl aliquots and stored at −80° C. in BHI broth with FCS for no more than 1 month until use. Mice were anesthetized with 2.5% isofluorane in oxygen and a total of 10⁶CFU/10 μl of a suspension of bacteria in sterile PBS was administered intranasally using a micropipette. At predetermined time points, the animals were euthanized in a CO₂ chamber and various tissues dissected.

Nasopharynx-to-brain meningitis model

Mice were anaesthetised with a mixture of O2 and isofluorane and infected intranasally with 10⁸CFU/10 ul per mouse. Mice were periodically scored for clinical signs of disease and were culled at pre-determined times post-infection by cervical dislocation and tissues were removed aseptically for determination of viable counts. Blood samples were taken by cardiac puncture.

Mouse challenge studies in pneumonia and sepsis models

The pneumococcal isolates were streaked onto 5% blood agar plates. Single colonies were grown overnight in BHI, and then sub-cultured in BHI+20% serum for storage at −80° C. Inocula were administered to mice in sterile PBS vehicle solution at varying densities: in the pneumonia model, mice were anesthetized and infected intranasally with 10⁶ CFU/50 μl/mouse, while in the sepsis model, mice received an intravenous administration of 10⁶ CFU/100 μl/mouse. All experiments were conducted over a period of 72 hours monitoring.

Immunisation protocol.

The immunogenicity of a trivalent protein-based vaccine of the present invention+CpG chitosan formulations (PrPV) was assessed in C57BL/6 mice (1-week old infants vs 6-week-old adults) following intranasal immunisation. A volume of 10 μl of solution were administered intramuscularly on days 7, 14 and 21 to groups of n=10 infant mice/group. Adult mice were immunised in the same manner at weeks 6 (prime), 8 (boost 1) and 10 (boost 2). Control groups were administered with either PBS or a free antigen solution, i.e., antigen solution devoid of adjuvant. In preliminary experiments, optimal vaccine dosage (ranging from 1 to 10 μg/animal) was determined by measuring antibody responses at 2 weeks post-tertiary vaccination. In addition to the negative/placebo control group, the vaccine formulations were also evaluated against the currently licensed vaccine PCV-13 (tradename Prevenar13®).

Passive transfer studies.

Adult mice immunised with either vehicle or the vaccine PrPV formulation containing ABC-T+PavA+ZmpB adsorbed into CpG-Chitosan were culled by cardiac puncture, and serum (100-150 μl/mouse) were transferred into naive animals via intravenous administration. At 4 hours post-transfer, animals were challenged either intranasally (pneumonia model) or intravenously (sepsis model) with S. pneumoniae. Mouse survival and tissue viable counts were recorded.

Measurement of immune responses.

To characterise anti-pneumococcal adaptive immunity, antigen-specific serum antibody responses (total IgG, antigen specific IgG1, IgG2a) were determined using commercial sandwich ELISA assays (R&D systems). Cell-mediated immunity were also determined by measuring pneumococcal antigen-specific CD3+CD4+ T cell responses: single cell suspensions were prepared from mesenteric lymph nodes and restimulated ex vivo in the presence or absence of pneumococcal antigens (ABC-T+PavA+ZpmB). Cytokine responses were determined using intracellular staining kits (BD Biosciences) for Th1/Th2 and Th17 panels, with a particular focus on IFN-γ/IL-4 and IL-17.

Comparative Genomic Analysis

One-hundred and forty pneumococcal isolates, originating from Malawian adults and children with meningitis (n=70) vs. bloodstream infection without clinical meningitis (bacteraemia; n=70), were subjected to Illumina sequencing. The mapped genes were clustered and divided into three categories: orthologous genes shared by all isolates (core genes), orthologous genes shared between two or more isolates (accessory genes), and genes unique to one isolate. A total of 3,164 orthologous gene clusters were uncovered: 45% (1,428 core genes) were present in all of the study isolates while 1,612 (51%) were present in two or more isolates, and the remaining 124 genes (4%) did not group within a cluster. Unexpectedly, no difference between the core genome or accessory gene content of the 140 isolates was found. An approach built on probabilistic mixture (GLOOME) was taken in order to compare the evolutionary history of gene gain/loss events (unpublished). Here, a small subset of meningitis-specific genes was identified—namely, pneumococcal adherence virulence factor A (PavA, gene ID: 12889101); ATP-binding cassette/transporter permease (ABC-T, gene ID: 13695552); and Zinc metalloprotease B (ZmpB, NCBI gene ID: 7680834), amongst others. These genes became of interest on the basis of their low sequence homology with human proteins, their documented functions, and their surface expression pattern. Subsequently genomic analysis was expanded to a dataset comprised of over 25,0000 pneumococcal isolates. In line with the goal to design a vaccine formulation providing global coverage across ages, a set of isolates comprising 44 distinct pneumococcal serotypes, originating from both developed and developing countries, spanning 6 continents (Asia, Africa, North America, South America, Europe, Australia) and 36 different countries were evaluated.

By virtue of their significant role in pneumococcal virulence, it was expected that the three pneumococcal proteins may also be the most variable in sequences. For instance, this has been demonstrated in the case of pneumolysin, for which 21 distinct allelic variants have so far been identified. Hence the degree of gene sequence variability (allelic variants) was determined for each identified candidate gene and their genetic relationship assessed, prevalence at the global level, serotype-specific association, and select the most prevalent variant form.

Statistical Analysis

For comparison of multiple groups, the statistical significance of endpoints was evaluated by one-way ANOVA followed by Tukey's multiple comparisons post hoc test when data were normally distributed. When data were not normally distributed, statistical significance of endpoints was evaluated by Kruskal-Wallis test followed by Dunn's multiple comparisons post hoc test. For comparison of two groups, the unpaired two-tailed Student's t test was used. Data are presented as means f SEM in bar graphs. Log-rank (Mantel-Cox) test was used to analyse the pneumococcal challenge survival curves. Results with p-values less than 0.05 were considered significant with * p-value<0.05, **p-value<0.01, ***p<0.001, ****p<0.0001). Statistical analysis was performed with the aid of Graph Pad Prism.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference. The following examples are illustrative and are not intended to limit the scope of the invention.

### Example 1

The levels of specific IgG antibodies to ZmpB, AbcT and PavA were determined by enzyme immunoassay in sera obtained from n=12 children (aged between 2 and 7 years), n=12 healthy adults (aged 18-45 years), n=12 adult patients presenting pneumococcal community acquired pneumonia (CAP), n=12 adults living with HIV, and n=12 elderly patients aged 65-75 years of age. High-binding microtiter plates (Corning) were coated with 5 ug/ml of total proteins (either as single AbcT, PavA, ZmpB or in combination AbcT+PavA+ZmpB) in carbonate-bicarbonate buffered saline (50 mM, pH 9.2) and incubated at 4° C. overnight. Blocking was done with 1% w/v BSA in PBS. Sera were serially diluted 1:20-1:40, 960 in 0.5% BSA w/v in PBS. The secondary antibody used was the alkaline phosphatase-conjugated monoclonal anti-human IgG (Sigma, A2064-1ML). An OD of ≥0·04 (two standard deviations of the control serum) for all measurements was considered to be positive. Samples with undetectable anti-ZmpB or anti-AbcT/PavA were assigned a value equivalent to half the detection limit. The protein-specific IgG results are given semi-quantitatively based on OD values (OD units at 405 nm). These were calculated from the mean OD readings of triplicate samples after subtraction of the OD readings of serum negative control wells.

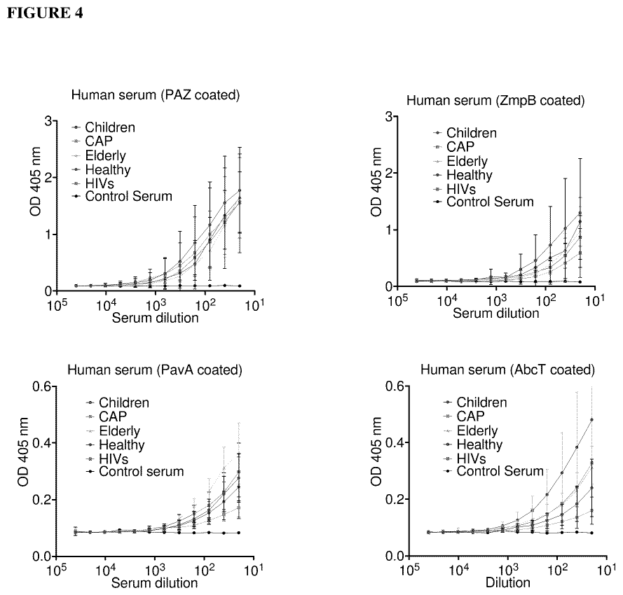

FIG. 4 shows the binding activity of human serum antibodies collected from healthy adult volunteers or from individuals susceptible to pneumococcal invasive diseases, namely, children, elderly and patients living with HIV. The results suggest that the 3 proteins described herein, i.e. ABC-T, PavA and ZmpB, contains antibody-binding regions (or B-cell epitope) recognised by human antibodies hence supporting their use as potent vaccine candidates.

### Example 2

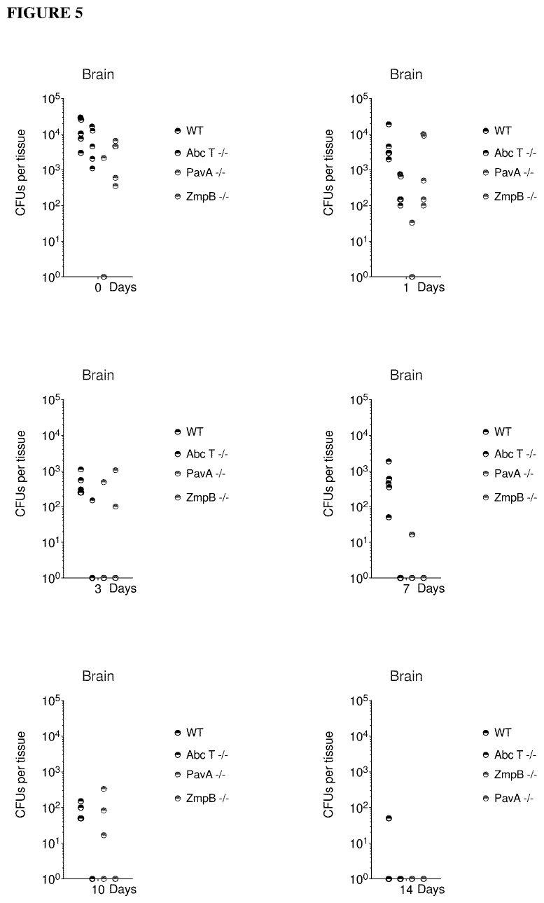

FIG. 5 shows the results from the validation of universally expressed conserved pneumococcal antigens in a meningitis mouse model. S. pneumoniae in 10 μl sterile saline solution, were applied to the nose or injected in the cisterna magna. 100 μL of the brain tissue homogenate was transferred to a well on a 96-well plate and ten-fold serial dilutions made in sterile PBS. 60 μL aliquots were spotted on blood agar plates containing 10 μg/ml gentamicin. Bacterial burden (CFU) from tissue homogenates enumerated from infected animals in a preclinical meningitis model at 0, 1, 3, 7, 10 and 14 days post-infection. These results show that when the expression of ABC-T, PavA and ZmpB proteins is null, the virulence i.e. capacity to spread and invade host tissues, of Streptococcus pneumoniae is significantly attenuated. These observations strongly support the use of these proteins as vaccine candidates.

### Example 3

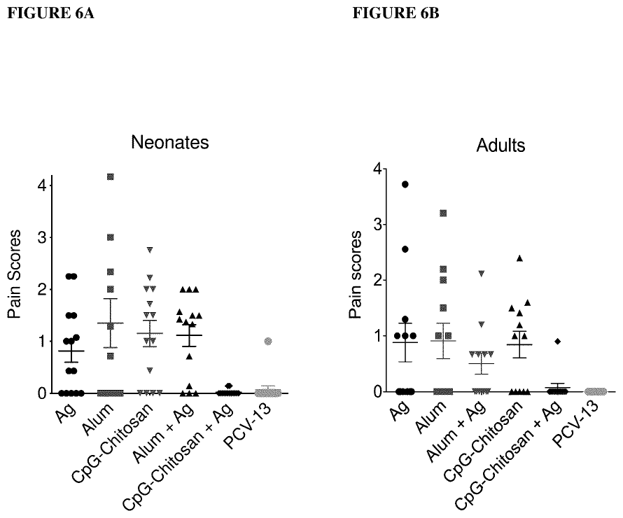

Mice were immunised with a mixture of the three antigenic determinants corresponding to SEQ ID NOS: 1, 2 and 3 of the present invention, either alone (Ag) or combined with Alum (Alum+Ag) or CpG-chitosan (CpG-Chitosan+Ag)/PrPV using a prime-boost regimen and challenged with 5·10⁵ CFU/mouse in 50 ul intranasally. Mice were monitored for the onset of signs of disease following and the clinical pain scores in C57BL/6 mice (FIG. 6A) adults and (FIG. 6B) neonates, (n=10 per group) were recorded.

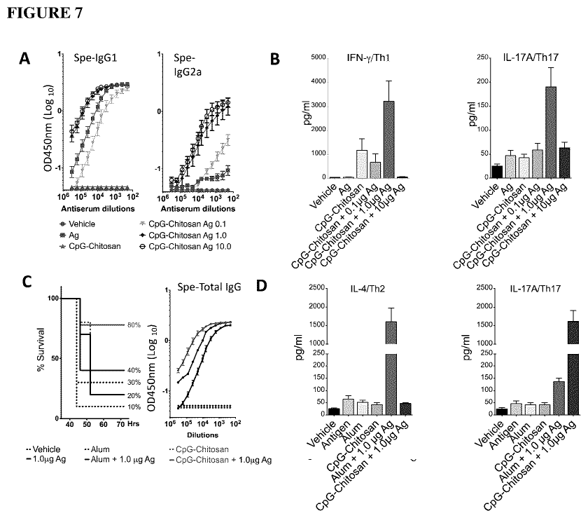

Antibody dose-response immunisation studies were conducted with mixtures of the three antigenic determinants corresponding to SEQ ID NOS: 1, 2 and 3 of the present invention (Ag) in various amounts, each in combination with a constant amount of CpG-Chitosan. Antigen-specific antibody (Spe-IgG1, Spe-IgG2a) levels in antiserum were measured from mice (n=10) vaccinated with prime+booster doses of recombinant protein antigens (Ag) adsorbed onto CpG-Chitosan at 0.1, 1.0 and 10 μg of antigens/mouse. Antiserum from PBS/vehicle vaccinated animals were included as controls. Results displayed as mean±SEM (FIG. 7A). Cell-mediated immune response was determined using mesenteric lymph nodes collected on day 35 post-immunisation upon ex vivo re-stimulation with PavA+ABC-T+ZmpB (500 μg/mL) for 72 hours. Enzyme-linked immunosorbent assays (ELISA) were conducted to determine IL-17 and IFN-γ cytokine production. (FIG. 7B).

Mice were vaccinated with PBS (Vehicle), adjuvant alone (Alum or CpG-Chitosan), or adjuvant+antigen (Alum+1.0 μg Ag; CpG-Chitosan+1.0 μg Ag). At 35 days post-vaccination, mice were challenged intranasally with 5×10⁵ CFU of hypervirulent pneumococcal serotype 1 clinical isolate and culled upon development of disease symptoms. Vaccine antigen-specific responses in mesenteric lymph nodes collected on day 35 post-immunisation were determined by ex vivo restimulation with PavA+ABC-T+ZmpB (500 μg/mL) for 72 hours. Enzyme-linked immunosorbent assays (ELISA) were conducted to determine IL-17 (Th1) and IL-4 (Th2) cytokine production. Mean±SEM. *p<0.05;**:p<0.01 Kruskal-Wallis with Dunn's post-test (vs. vehicle (FIGS. 7C and 7D). Vaccination with PrPV formulation provides enhanced disease protection compared to conventional Alum adjuvant.

### Example 4

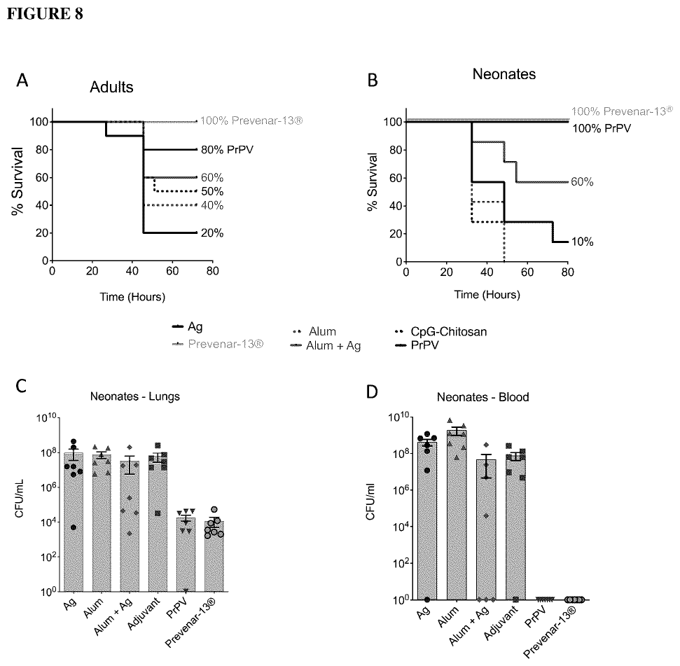

The protective efficacy of PrPV was compared against PCV-13 in models of pneumococcal sepsis and pneumonia in adult (FIG. 8A) and neonate (FIG. 8B) mice. Mice were immunised with recombinant ZmpB, ABC-T and PavA combined with Alhydrogel (“Alum+Ag”), and challenged with S. pneumoniae 5×10⁵ CFU i.n. Survival of animals was monitored for 7 d after challenge. Numbers of surviving mice are plotted as a percentage of the total (n=10 mice per group). FIG. 8C shows bacterial densities in lung of individual neonate mice at day 3 after challenge. Blood was collected via cardiac puncture and spotted onto blood agar plates for CFU viable counts (FIG. 8D). For all experiments, naïve and adjuvant only-immunised mice served as negative controls.

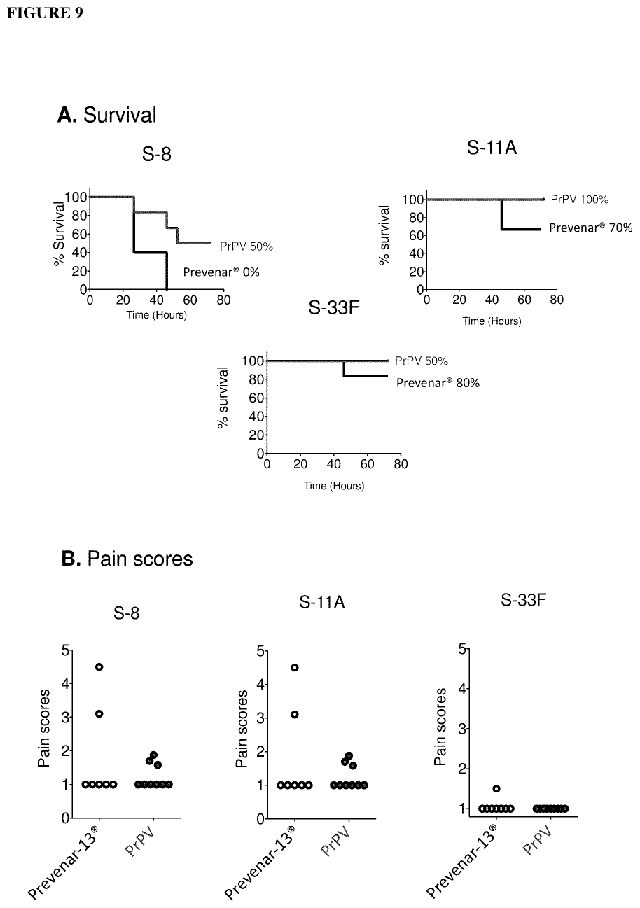

In a further experiment, mice were immunised with recombinant ZmpB, ABC-T and PavA corresponding to SEQ ID NOS: 1, 2 and 3 combined with CpG-Chitosan (PrPV) and subsequently challenged with S. pneumoniae 1×10⁶ CFU i.n. Survival of animals was monitored for up to 5 days post-challenge. Numbers of surviving mice were plotted as a percentage of the total (n=10 mice per group). Non-vaccine pneumococcal serotypes—e.g. 8, 11A, and 33F, which have been reported as emerging serotypes in England and Wales—were tested to assess the breadth of protective coverage of the vaccine formulation. PCV-13 was used as the comparator, n=10 mice per group. Mouse survival rates (FIG. 9A), and clinical pain scores were monitored (FIG. 9B). These results show that the PrPV formulation of the present invention provides better immune protective efficacy against non-vaccine serotypes compared to commercial vaccine PCV-13 (Prevenar-13).

### Example 5

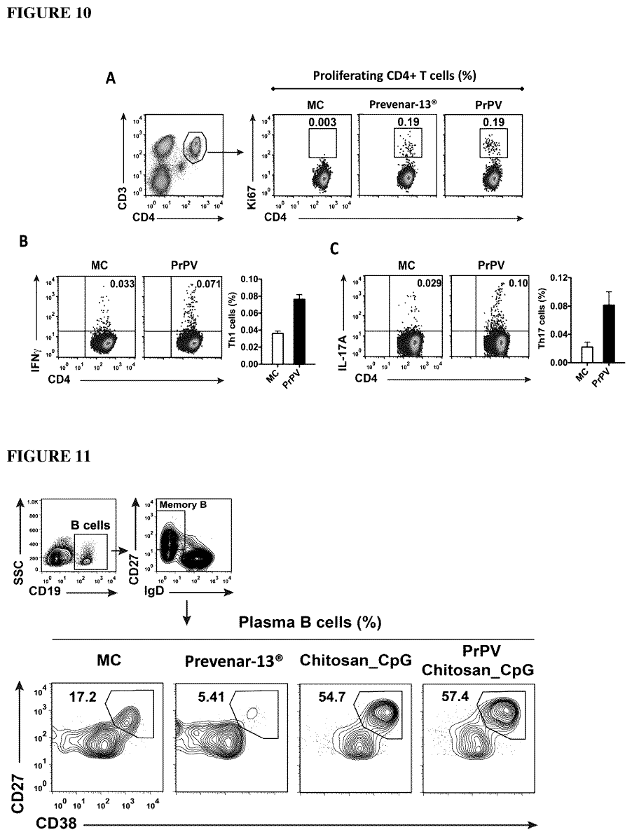

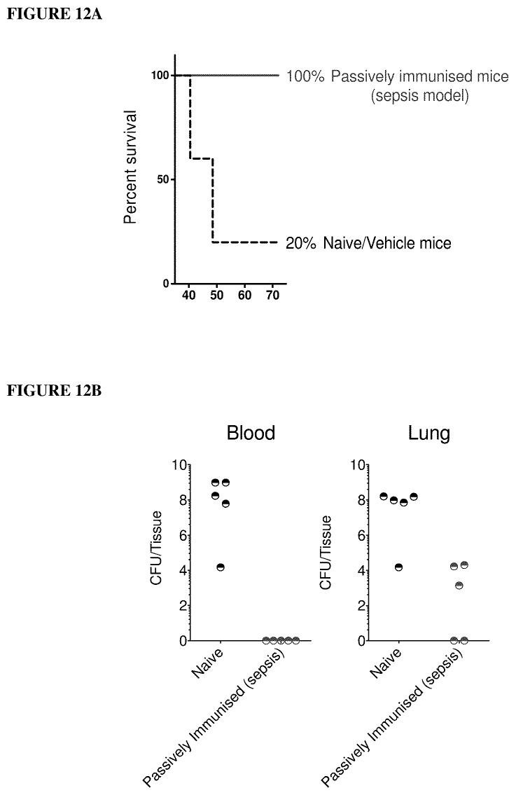

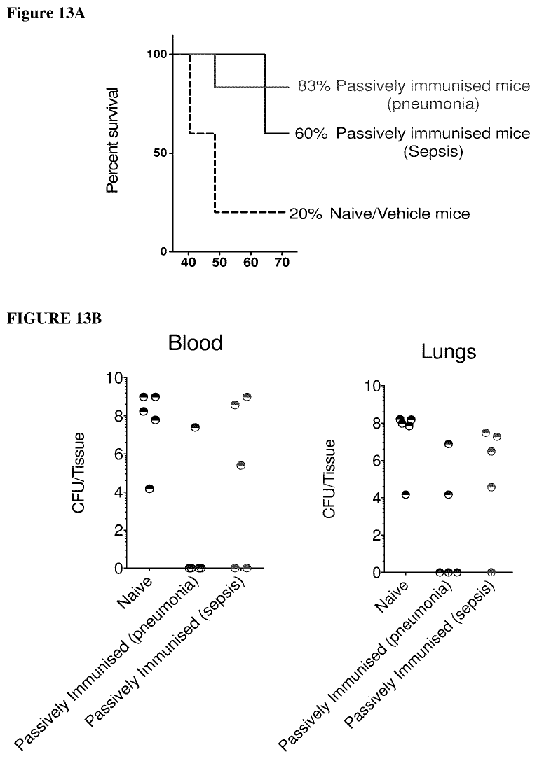

Isolated human PBMCs were stimulated with PCV-13 (1:50) and PrPV respectively for 72 hours, and T cell responses were examined by intracellular staining. FIG. 10 shows (A) CD3⁺CD4⁺ T cells were gated out to illustrate Ki67⁺ proliferating T cells in the rectangular gate with percentages indicated on top. Percentages of IFNγ⁺CD4⁺ Th1 (B) and IL-17A⁺CD4⁺ Th17 cells (C) were shown in the top right quadrants of the dot plots. Data from two individual donors were summarised in the bar figures. MC: media control. Results indicate that PrPV activates CD4* T cell responses in human PBMCs. In a similar experiment, isolated human PBMCs were stimulated with PCV-13 (1:50), Chitosan_CpG and PrPV respectively for 7 days, followed by the detection of plasma B cell activation. Percentages of plasma B cells (gated as CD27^{high}CD38^{high}) within memory B cells (CD19⁺CD27⁺IgD⁻) are shown in the bottom contour plots (FIG. 11). Data is representative of two individual donors. These results show that PrPV activates plasma B cells in human PBMCs.

### Example 6

Adult mice (n=10/group) immunised with either vehicle or the PrPV formulation (containing ABC-T/PavA/ZmpB corresponding to SEQ ID NOS: 1, 2 and 3 adsorbed into CpG-Chitosan) were culled by cardiac puncture, and serum (100-150 μl/mouse) were transferred into naive animals via intravenous administration. At 4 hours post-transfer, animals were challenged intravenously (sepsis model) with a disease-causing dose of S. pneumoniae. Mouse survival times (FIG. 12A) and CFU viable counts in blood and lungs (FIG. 12B) were recorded. These results show that serum from PrPV-immunised mice confers passive immunity in naïve mice.

Human PBMCs from healthy volunteers were isolated from whole blood by Ficoll-Paque Plus using density gradient separation and cultured for 7 to 8 hours in the presence of the PrPV composition. Supernatants were collected, concentrated using Vivaspin concentrators (10K MWCO) and administered intravenously to adult naïve mice. At 4 hours post-transfer, animals (n=5/group) were challenged either intranasally (pneumoniae model) or intravenously (sepsis model) with S. pneumoniae. Mouse survival times (FIG. 13A) and CFU viable counts in blood and lung (FIG. 13B) were recorded. These results show that concentrated supernatants from PrPV-stimulated human PBMC cultures confers passive immunity in naïve mice.

### Example 7

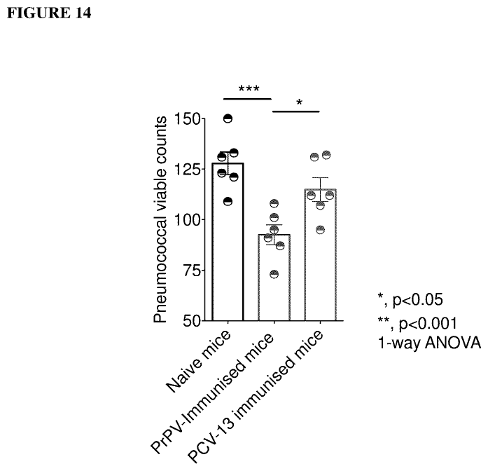

Adult mice (n=10/group) immunised with either vehicle or the PrPV formulation were culled by cardiac puncture, and serum (100-150 μl/mouse, Diluted 1:16) as collected for use in a functional opsonophagocytosis killing assay (OPK assay). OPKs were performed to test the ability of vaccinated mouse antiserum to opsonise viable pneumococci and mediate killing. A reference opsonophagocytic assay was used whereby reaction mixture consisting of heat-inactivated serum obtained from vaccinated vs. unvaccinated mice, differentiated HL-60 cells or freshly isolated human neutrophils, pneumococci, and baby rabbit complement were prepared. Control reactions lacking complement and/or antibody, effector cells or all components except pneumococci were run in parallel and incubated at 37° C. while shaking. The results show that serum from PrPV-immunised mice promotes in vitro opsonization of pneumococci and outcompete licensed PCV-13 vaccine (FIG. 14).

### Example 8

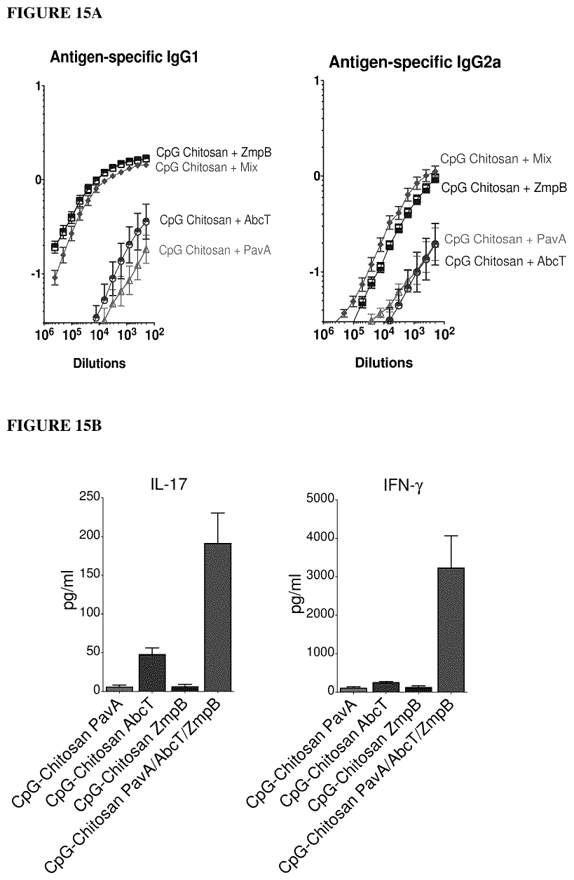

Antibody dose-responses for single proteins in combination with CpG-Chitosan versus the PrPV composition were studied. Antigen-specific antibody (Spe-IgG1, Spe-IgG2a) levels in antiserum measured from mice (n=10) vaccinated with prime+booster doses of the single protein or PrPV composition (labelled “CpG Chitosan+Mix) at 1.0 μg/mouse, were measured. Results displayed as mean f SEM (FIG. 15A). Cell-mediated immune response upon vaccination with single protein vs. mixed protein formulations was also studied. Vaccine antigen-specific responses in mesenteric lymph nodes collected on day 35 post-immunisation were determined by ex vivo re-stimulation with PavA, ABC-T or ZmpB (500 μg/mL) for 72 hours. Enzyme-linked immunosorbent assays (ELISA) were conducted to determine IL-17A (Th17) and IFN-gamma (Th1) cytokine production. (FIG. 15B). These results show that vaccination with mixed protein formulation present enhanced immunogenicity compared to single protein adjuvanted CpG formulations.

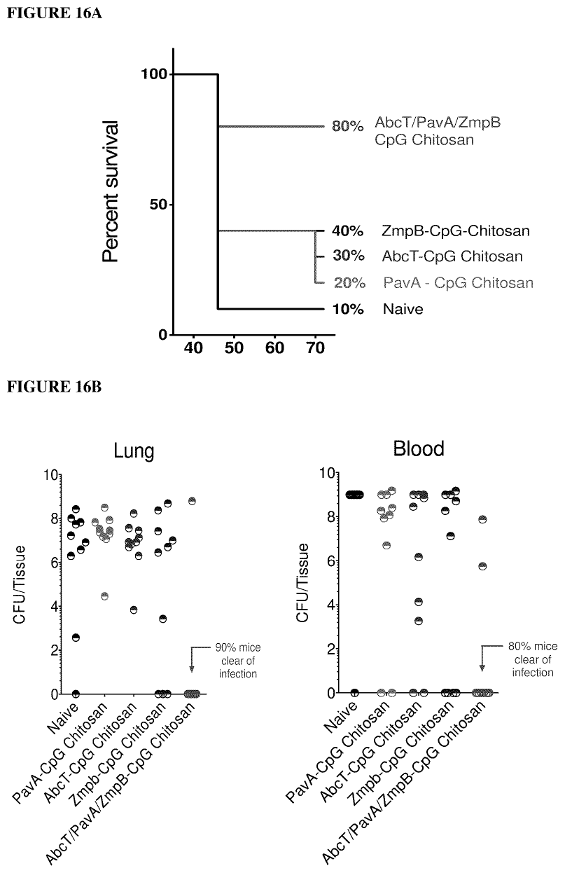

Adult mice (n=10 per group) were vaccinated with PBS (Vehicle), single antigen plus CpG-Chitosan or PrPV. At 35 days post-vaccination, mice were challenged intranasally with 5×10⁵ CFU of hypervirulent pneumococcal serotype 1 clinical isolate and culled upon development of disease symptoms. FIG. 16A shows the survival curve. The viable count of bacteria in blood and lungs was determined at the pre-chosen intervals after intranasal infection (FIG. 16B). Tissue samples were homogenised using an IKA T10 homogeniser. Viable counts in lung cell suspension and in blood samples were determined by serial dilution in sterile PBS and plating on blood agar containing 5% (v/v) defibrinated horse blood (Oxoid) and 10 ug/ml gentamicin (sigma). Once dry, plates were incubated in 5% (v/v) CO2 at 37° C. overnight and bacterial colony numbers assessed the following day. These results show that vaccination with a mixed protein formulation of the present invention provides enhanced disease protection compared to single protein adjuvanted formulation.

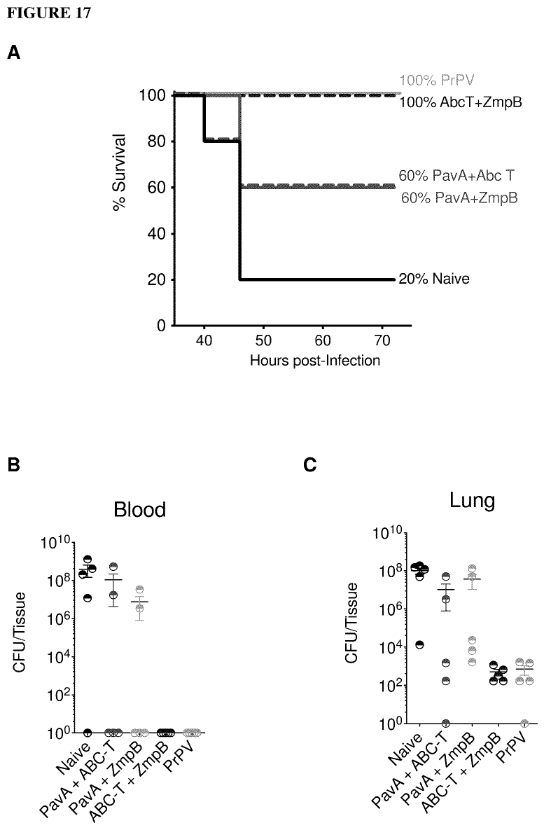

Adult mice (n=5 per group) were vaccinated with PBS (Vehicle), or adjuvant+antigen (CpG-Chitosan+1.0 μg Ag in paired-protein formulations). The paired protein formulations were proteins corresponding to SEQ ID NOS: 1, 2 and 3 (PavA+ABC-T or ABC-T+ZpmB or PavA+ZmpB). At 35 days post-vaccination, mice were challenged intranasally with 5×10⁵ CFU of hypervirulent pneumococcal serotype 1 clinical isolate and culled upon development of disease symptoms. FIG. 17A shows the survival curve. The viable count of bacteria in blood (FIG. 17B) and lungs (FIG. 17C) was determined at the pre-chosen intervals after intranasal infection. Tissue samples were homogenised using an IKA T10 homogeniser. Viable counts in lung cell suspension and in blood samples were determined by serial dilution in sterile PBS and plating on blood agar containing 5% (v/v) defibrinated horse blood (Oxoid) and 10 ug/ml gentamicin (sigma). Once dry, plates were incubated in 5% (v/v) CO2 at 37° C. overnight and bacterial colony numbers assessed the following day. These results show that vaccination with paired protein formulations induces a protective efficacy in the order PavA+ZmpB, PavA+ABC-T, ABC-T+ZmpB.

### Example 9

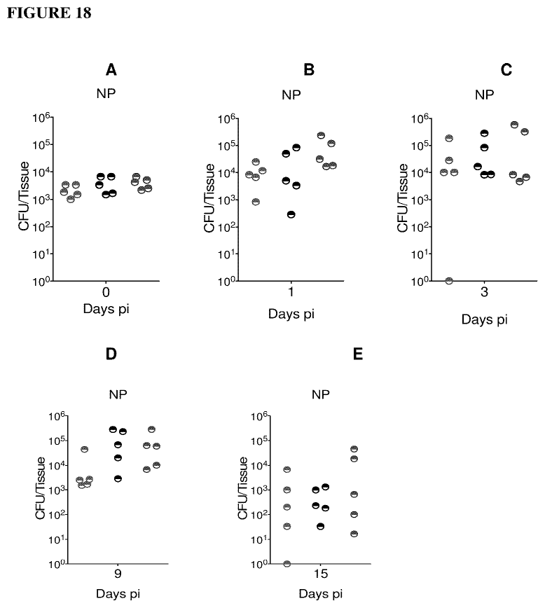

The effect of an immunological composition of the present invention (PrPV) on pneumococcal colonization and shedding was assessed in C57BL/6 mice following intramuscular immunisation. A volume of 10 μl of solution was administered intramuscularly on postnatal days 7, 14 and 21 to n=10 infant mice/group. Control groups were administered with either adjuvant alone or a free antigen solution, i.e. antigen solution devoid of adjuvant. Two weeks after the second booster, inocula were intranasally administered to mice in sterile PBS vehicle solution at 10⁵ pneumococcal CFU/10 μl/mouse. Bacterial densities (through tissue homogenization and nose tapping) were determined over a monitoring period of 15 days, (FIG. 18). Elimination of the pneumococcus from the human nasopharynx can have consequences that could be deleterious. A reasonable approach therefore, is to develop vaccines that allow the pneumococcus to remain in the nasopharynx but prevent the dissemination to body sites that result in an infection. The PrPV composition of the present invention provides protective efficacy against pneumococcal disease but does not impact on pneumococcal ecology within the mouse nasopharynx.

### Example 10

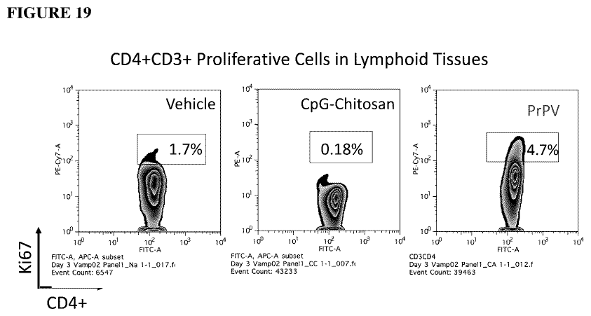

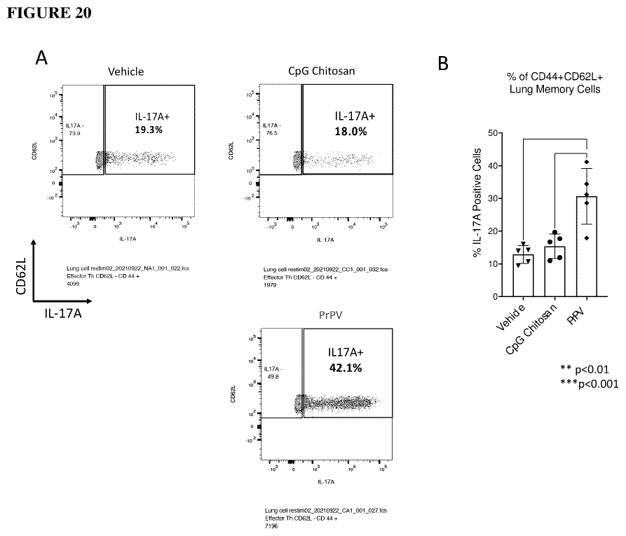

The immunological response induced by PrPV in mice was assessed by flow cytometry. A volume of 10 μl of PrPV solution was administered intramuscularly on postnatal days 7, 14 and 21 to n=10 infant mice/group. Additional groups of mice were administered with either adjuvant alone (“CpG-Chitosan”) or with vehicle control solution. Two weeks after the second booster, mice were primed with 2·10⁵ CFU pneumococcal serotype 1 in 50 μl/mouse. Immune cell proliferation measured in mesenteric lymph nodes at 3 days post-pneumococcal infection is shown in FIG. 19. The results show that a composition of the present invention (PrPV) is potent at inducing proliferative responses (as measured by percentage of CD3+CD4+Ki-67 positive cells) in the lymphoid structures of PrPV-immunised mice.

### Example 11

Adults Mice (n=5 per group) were vaccinated with PBS (Vehicle), CpG-chitosan adjuvant alone (“CpG-Chitosan”), or PrPV (i.e. CpG-Chitosan+1.0 μg SEQ ID NO:1, 1.0 μg SEQ ID NO:2+1.0 μg SEQ ID NO:3/mouse). At 35 days post-vaccination, mice were challenged intranasally with 5×10⁵ CFU of hypervirulent pneumococcal serotype 1 clinical isolate and culled 24 hours later. Vaccine antigen-specific responses in lung single cell suspension collected at the experimental endpoint were determined by ex vivo re-stimulation with PavA, ABC-T or ZmpB (500 μg/mL) for 24 hours. Briefly, mouse lungs were dissected out and transferred into a bijou tube containing Hank's buffer (HBSS). Any remaining tracheal tissue was carefully removed, and lung tissue was cut into small pieces using a Petri dish. A digestion solution containing collagenase at 300 U/ml and DNase I at 75 ug/ml was added to the lung pieces (2 ml/mouse) and incubated at 37C for 40 min-1 hr. The digested tissue was passed through a cell strainer, centrifuged and the pellet was resuspended in 5 ml of 1× RBC lysis buffer (4 min incubation) and cells were counted and seeded at a density of 4×10⁶ cells/ml. The derived single-cell suspensions were stained with anti-CD62L, anti-CD44 or anti-IL-17A antibodies conjugate to their respective fluorophores, and analyzed by flow cytometry. FIG. 20A shows representative dot plots of each group, namely naive, antigens, CpG-chitosan (adjuvant alone) and PrPV—the upper right quadrant indicates the percentage of CD62L+CD44+IL17A-positive cells. FIG. 20B shows that the percentage of CD62L+CD44+IL17A-positive cells in PrPV-immunised mice is significantly higher than all the other groups (**<p<0.01; ***p<0.001). These results suggest that vaccination with PrPV leads to an IL-17A-mediated protective immunity, which is significantly higher compared to the vehicle or adjuvant only groups.

### Example 12

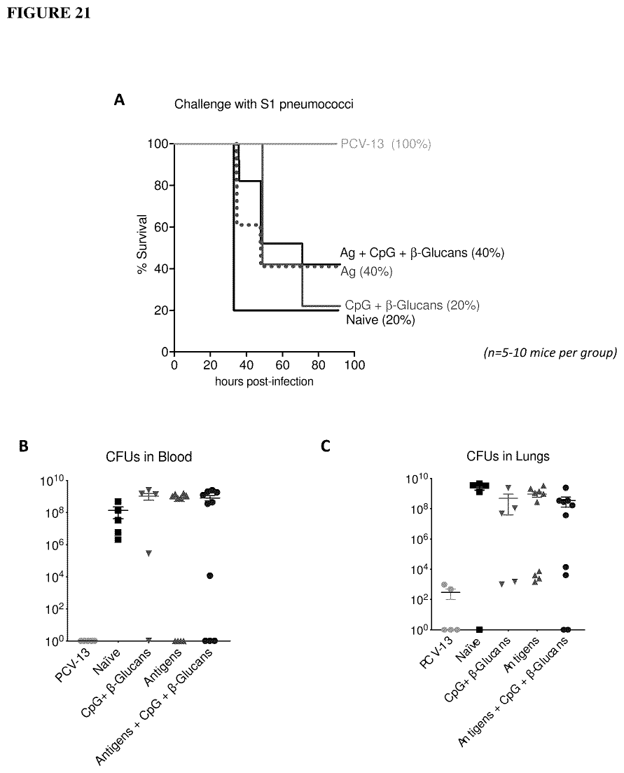

The protective efficacy of the antigens of the present invention was tested upon co-formulation with beta-glucans (β 1,3/1,6-D-glucan, IRI 1501, isolated from Saccharomyces cerevisiae). Adult mice were vaccinated with PBS (Vehicle), adjuvant alone (beta glucan-based formulation supplemented with CpG or not), or adjuvant+a mixture of ZmpB and PavA antigens corresponding to SEQ ID NOS: 3 and 2 (beta glucan-based adjuvant+1.0 μg Ag supplemented or not with CpG). At 35 days post-vaccination, mice were challenged intranasally with 5×10⁵ CFU of hypervirulent pneumococcal serotype 1 clinical isolate and culled upon development of disease symptoms. Survival curves (FIG. 21A) and viable counts (CFUs) were determined in blood (FIG. 21B) and lung tissues (FIG. 21C). A protective efficacy of 40% was obtained in mice administered with either (antigens alone) or (antigen+CpG+beta-glucans), hence suggesting that a beta-glucan-based adjuvanted formulation does not interfere with the protective efficacy conferred by the antigens of the present invention, and may be developed into a potent pneumococcal vaccine upon further optimisation e.g. dosage, immunisation schedule, and/or reformulation with other immunostimulatory compounds.

1. An immunogenic composition comprising at least two antigenic determinants, wherein the antigenic determinants are derived from at least two proteins selected from ABC-T, PavA and ZmpB of a Streptococcus pneumoniae bacterium.

2. An immunogenic composition comprising a genetic construct or constructs encoding at least two antigenic determinants, wherein the antigenic determinants are derived from at least two proteins selected from ABC-T, PavA and ZmpB of a Streptococcus pneumoniae bacterium.

3. An immunogenic composition comprising at least one antigenic determinant and a genetic construct or constructs encoding at least one different antigenic determinant, wherein the antigenic determinants are derived from at least two proteins selected from ABC-T, PavA and ZmpB of a Streptococcus pneumoniae bacterium.

4. An immunogenic composition as claimed in any of claims 1 to 3, wherein the antigenic determinants are derived from all three of the proteins ABC-T, PavA and ZmpB of a Streptococcus pneumoniae bacterium.

5. An immunogenic composition as claimed in any of the preceding claims, wherein the antigenic determinant derived from ABC-T comprises or consists of the protein sequence SEQ ID NO:1, or an immunologically effective fragment thereof or an immunologically effective analog of the sequence or fragment thereof, and/or
wherein the antigenic determinant derived from PavA comprises or consists of the protein sequence SEQ ID NO:2, or an immunologically effective fragment thereof or an immunologically effective analog of the sequence or fragment thereof; and/or
wherein the antigenic determinant derived from ZmpB comprises or consists of the protein sequence SEQ ID NO:3, or an immunologically effective fragment thereof or an immunologically effective analog of the sequence or fragment thereof.

6. An immunogenic composition as claimed in any of the preceding claims further comprising an immunostimulatory agent, preferably wherein the immunostimulatory agent is an adjuvant.

7. An immunogenic composition as claimed in claim 6, wherein the adjuvant comprises one or more materials selected from materials having a stimulatory effect on Toll-Like Receptors (TLR), cytosolic pattern recognition receptors (PRRs) such as nucleotide-binding oligomerization domain (NOD)-like receptors (NLRs), C-type lectin receptors (CLRs), nucleic acid-sensing receptors such as retinoic acid-inducible gene I (RIG-I), or nutrient sensors such as mTOR and GCN2; materials acting on at least one of the following pathways: MyD88/TLR-signalling pathways, cGAS-stimulator of interferon genes (STING) pathway, NF-κB pathway, any stress, cell death and tissue damage pathways (such as necrosis/necroptosis, apoptosis, autophagy) resulting in release of damage-associated molecular patterns (DAMPs) such as nucleic acids, uric acid, ATP and proteins that activate the innate immune system, any signalling pathways resulting in immune cell recruitment or epigenetic changes that maintain the innate immune system at an alarming state for extended periods i.e. a memory-like state; materials having the property to induce inflammasome activation ensued by cell differentiation to a CD4+Th2 (interleukin-4), Th1 (Interferon-gamma)- or Th17(IL17A)-mediated immunity; immune potentiators; polysaccharide-based materials acting on the IL-1β, CLRs and TNF-α signalling pathways); delivery systems and mucosal adjuvants.

8. An immunogenic composition as claimed in claim 6 or claim 7, wherein the adjuvant comprises one or more materials selected from bacterial proteins and/or polysaccharide materials such as capsules and analogs thereof, toxin/toxoid and analogs thereof, TLR ligands and analogues thereof, agonists such as Pam3CSK4, Pam2CSK4, MPLA (LPS derivative), CpG, PolyI:C, CpG motifs; microparticles and nanoparticles such as chitosan or beta-glucans, flagellin, poly(lactic-co-glycolic acid) or PLGA-poly-1-lysine/poly-gamma-glutamic acid (PLGA-PLL/gammaPGA), or made of lipid-based backbone co-formulated with any other immunostimulatory agents such as monophosphoryl lipid A (MPLA), polyethylene glycol (PEG), oligomannose, Poly(I:C), extracellular vesicles (EV), such as outer membrane vesicles (OVM); oil-based encapsulation or emulsion-based systems, including water-in-oil, oil-in-water and thermo reversible oil-in-water emulsions such as Montanide ISA-51, Montanide ISA-720 and analogs thereof, alum or aluminium salt adjuvants, liposomes, virosomes, archeosomes, outer-membrane vesicles, niosomes, saponins, and immunostimulating complexes (ISCOMs), polymeric particles, cytokines including proinflammatory cytokines such as IFN-γ, IL-1, IL-2, IL-4, IL-12, IL-17A/F, GM-CSF and MPI, virus-like particles (VLPs), bacterial components such as detoxified variant of LPS such as Monophosphoryl lipid A (MPL), muramyl dipeptide (MDP lipophilic and hydrophobic), QS21, PLGA, CT, liposome-based cationic adjuvant formulation e.g. CAF 01-09 (composed of Span80, polyoxyethylene cetyl-stearylether, mannitol, squalene) and analogs composed of cholesterol, phosphatidylcholine and phosphatidylserine, or alpha-Gal ceramide, NOD-like receptor protein 3 (NLRP3) inflammasome, Apoptosis-associated speck-like protein containing a CARD (ASC), NLR-family CARD domain-containing protein 4 (NLRC4) inflammasome, Absent in melanoma 2 (AIM2) inflammasome, dsRNA: Poly(I:C), Poly-IC:LC, Monophosphoryl lipid A (MPL), LPS, Flagellin, Imidazoquinolines: imiquimod (R837), resiquimod (848), CpG oligodeoxynucleotides (ODN), Muramyl dipeptide (MDP), Saponins (QS-21), bacterial molecules or derivatives including polysaccharide capsules and analogs thereof, toxin/toxoid and analogs thereof, unmethylated DNA (CpGs) and analogs thereof, cytokines such as IL-2, IL-12, TNF-α, and granulocyte-macrophage colony-stimulating factor (GM-CSF), chemokines such as RANTES (regulated on activation, normal T cell expressed and secreted), macrophage inflammatory protein (MIP)-1α, costimulatory or adhesion molecules such as CD80, lymphocyte function-associated antigen-3 and polyarginine tails, polysaccharide-based materials with properties similar to those presented by chitosan, such as CpG-delta Inulin, cochleates, virus-like particles (VLP), microparticulates such as virosomes, PLA (polylactic acid), PLG (poly[lactide-coglycolide]), Cholera toxin (CT) derivatives, and Heat-labile enterotoxin (LTK3 and LTR72).

9. An immunogenic composition as claimed in any of claims 6 to 8, wherein the adjuvant comprises one or more materials selected from TLR agonists, agents capable of inducing a CD4+ T-cell mediated immune response (particularly with a Th1- and/or Th17-differentiated profile), oil-in-water emulsions, water-in-oil emulsions, agents acting on the MyD88/TLR-signalling pathway, agents acting on the cGAS-stimulator of interferon genes (STING) pathway and particulate polysaccharide materials; preferably wherein the adjuvant comprises a combination of a TLR agonist and an agent capable of acting on the MyD88/TLR-signalling pathway or the cGAS-stimulator of interferon genes (STING) pathway; for example a combination of CpG with chitosan and/or beta glucan.

10. An immunogenic composition as claimed in any of the preceding claims further including one or more additional antigenic determinants from a Streptococcus pneumoniae bacterium that is encapsulated or conjugated selected from the group comprising pneumococcal surface protein A (PspA), pneumococcal choline-binding protein A (PcpA), secreted 45-KDa protein Usp45-hydrolase (PcsB), serine/threonine protein kinase (StkP), peptide permease enzyme, manganese ABC transporter (PsaA), pneumolysin D (PlyD), pneumolysin toxoid (dPly), choline-binding proteins A (CbpA) and D (CbpD), histidine triad protein D (PhtD), histidine triad protein E (PhtE), histidine triad protein A (PhtA), histidine triad protein B (PhtB); pneumococcal serine-threonine uptake protein A (PiuA), protein cell wall separation (PcsB), pneumococcal serine-threonine kinase (StkP) and analogs, plasmin and fibronectin-binding protein A (PfbB), SP0148, SP1912 and SP2108, SP0785, SP1500 and SP2216.

11. An immunogenic composition as claimed in any of the preceding claims that is a liquid, suspension, tablet or spray or is lyophilized.

12. An immunogenic composition as claimed in any of the preceding claims that is delivered by means of the group comprising virus-like particles (VLPs), outer-membrane vesicles (OMVs), liposomes, immune stimulating complexes (ISCOMs), polymeric, non-degradable, biodegradable materials, natural polymeric compounds, starch, alginates, cellulose, biosynthetic materials, Poly beta-hydroxybutyrate (PHB), co-polymers, Polylactic acid (PLA), polyurethane, Poly(lactic-glycolic acid) (PLGA), Polymethyl methacrylate resin (PMMA), probiotics, Lactobacilli and Bifidobacteria species; viral vector systems; modified Vaccinia virus Ankara (MVA), poxviruses, adenoviruses and bacteriophages.

13. A vaccine comprising an immunogenic composition according to any of claims 1 to 5.

14. A vaccine as claimed in claim 13, including one or more of the features of any of claims 6 to 12.

15. A method of treating or preventing a Streptococcus pneumoniae bacterium infection in an individual comprising administering a sufficient amount of the immunological composition of any of claims 1 to 12 or the vaccine of claims 13 and 14 to treat or prevent the Streptococcus pneumoniae bacterium infection in an individual in need thereof.

16. A method of immunising against a Streptococcus pneumoniae bacterium infection in an individual comprising administering an effective amount of the immunological composition of any of claims 1 to 12 or the vaccine of claims 13 and 14 to an individual in need thereof.

17. A method as claimed in claim 15 or claim 16 wherein the immunological composition or vaccine is administered orally, nasally, by inhalation or by injection.

18. A method as claimed in claim 17, wherein the administration is by a route selected from the group comprising intravenous, intraperitoneal, intramuscular, intracavity, subcutaneous intradermally, intranasally and inhalation.

19. An immunological composition according to any of claims 1 to 12 or a vaccine according to claim 13 or claim 14 for use in the treatment or prevention of a Streptococcus pneumoniae bacterium infection in an individual and/or for immunising against a Streptococcus pneumoniae bacterium infection in an individual and/or diagnosing or screening for a Streptococcus pneumoniae bacterium infection in an individual.

20. A kit comprising an immunogenic composition according to any of claims 1 to 12 or a vaccine according to claim 13 or claim 14, and means to administer the immunogenic composition or vaccine to an individual.

21. A method of treating or preventing a Streptococcus pneumoniae bacterium infection in an individual or immunising against a Streptococcus pneumoniae bacterium infection in an individual comprising administering a sufficient amount of at least one antigenic determinant derived from a protein selected from ABC-T, PavA and ZmpB of a Streptococcus pneumoniae bacterium and simultaneously or subsequently administering a sufficient amount of least one antigenic determinant derived from a protein selected from a different one of ABC-T, PavA and ZmpB of a Streptococcus pneumoniae bacterium.


SEQUENCE LISTING (6 sequences)

SEQ ID NO: 1 - protein, 359 aa, Streptococcus pneumoniae
MDGIAKEWGFASRSAYSRDILAMESFSTYIGNQFTRTEKLQTQTGNDSMKFNYNGISQFSSVRNRSSSSTLDKLGFKSSGTNLNLRYANNSILADSLFGIQYNISDSPIDKYGFKDIYQKDNLTLYENQYSLPIAVASQSVYNDVKFNEHTLDNQASFLNQLANVNFDYFSPIPYEKTEKIENTNDLISVTSSSNEDAAIQYQIEVPENSQVYLSFINLHFSNDKQKKVDILVNGEKKTFTTDNVFSFFNLGYTKEKKTFNINVSFPGNSQVSFESPTFYRLDTKTFTEAIQKIKEQPVTVSTSKNKVFATYDVQQDTSIFFTIPYDKGWSAYQDGKKIEIKQAQTGFMKVDIPKGGSG

SEQ ID NO: 2 - protein, 554 aa, Streptococcus pneumoniae
MSFDGFFLHHIVEELRSELVNGRIQKINQPFEQELVLQIRSNRQSHRLLLSAHPVFGRIQLTQTTFENPAQPSTFIMVLRKYLQGALIESIEQVENDRIVEITVSNKNEIGDHIQATLIIEIMGKHSNILLVDKSSHKILEVIKHVGFSQNSYRTLLPGSTYIAPPSTKSLNPFTIKDEKLFEILQTQELTAKNLQSLFQGLGRDTANELERILVSEKLSAFRNFFNQETKPCLTETSFSPVPFANQVGEPFANLSDLLDTYYKDKAERDRVKQQASELIRRVENELQKNRHKLKKQEKELLATDNAEEFRQKGELLTTFLHQVPNDQDQVILDNYYTNQPIMIALDKALTPNQNAQRYFKRYQKLKEAVKYLTDLIEETKATILYLESVETVLNQAGLEEIAEIREELIQTGFIRRRQREKIQKRKKLEQYLASDGKTIIYVGRNNLQNEELTFKMARKEELWFHAKDIPGSHVVISGNLDPSDAVKTDAAELAAYFSQGRLSNLVQVDMIEVKKLNKPTGGKPGFVTYTGQKTLRVTPDSKKIASMKKSGSG

SEQ ID NO: 3 - protein, 1301 aa, Streptococcus pneumoniae
MDKLKYYQGYTLSTTMVYDRGEGEETEKLEDKQIQLDLKKVEIKNIKETSLMNVDAEGNETDKSLLSEKPTDVSQLYLRVTTHDNKVTRLAVSSVEEVVVDGKTLYKVVAKAPDLVQRRADDTLSEEYVHYFEKQLPKVNNVYYNFNELVKDMQANPMGEFKLGADLNAVNVKPAGKAYVMAKFRGTLSSVENHQYTIHNLERPLFNEAEGATLKNFNLGNVNINMPWADKVAPIGNMFKKSTLENIKVVGSVTGNNDVTGAVNKLDEANMRNVAFIGKINSLGDKGWWSGGLVSESWRSNTDSVYFDGDIVGNNSKFGGLVAKVNHGSNQWDVKQKGRLTNSVVKGTMTLKNHGQSGGLVHENYDWGWVENNISMMKVNNGEIMYGSGSIDGDPYFGFDYFKNNYYVKDVATGESTYKRSKQIQSISQAEADAKIANMGITANTFAIQDPVVNKLNRIIDRDSEYKAIQDYQETRNLAYRNLEKLQPFYNKEWIVNQGNKLTDESNLVKKTVLSVTGMKSGQFVTDLSSVDKIMIHYADGTKEEFGVSAISDSRVKQVKEYNVDDLGVVYTPNMVDKNRDSLITKVKEKLSSVALDSAEVKSITNNPASLYLEESFAEVRETLDKLVKSLLENEDHQLNSDEVAEKALLKKVEDNKAKIILALTYLNRYYGIDYDGLNFKHLMMFKPDFYGKTPSILDFLIRIGSAEKNLKGDRSLEAYREVIGGTIGKGELNGLLGYNMRLFTKYTDLNDWFIHAAKNVYVSEPETTTEDFKDKRHRIYDGLNNDVHGRMILPLLNLKKAHIFVISTYNTIAFSSFEKYGKNTEEERNAYKAEIDRVAKAQQRYLDFWSRLALPKVRNQLLKSQNSVPTPVWDNQVYVGLGGANRMGYGDGGRVVTPVRELFGPTDRWHQINWNMGAMAKIYERPWKDDQVYFMVTNMMEPFGISAFTHETTHVNDRMAYYGGDWHREGTDLEAFAQGMLQTPDKSTTNGEYGALGINMAYERKNDGEQLYNYDPEKLDSREKIDSYMKNYNESMMMLDYLEASAVIRQNLSDNSKWFKKMDKEWRTNADRNRLIGEPHQWDKLRDLTEEEKKLPIDSIDKLVENNFVTLHGMPKNGRYRTEGFDSSYQPVNMMAGVFGGNTSKSTVGSISFKHNAFRMWGYYGYENGFIPYVSNKLKGAANKENKGLLGDDFIIKKVSKNQFQNLEEWKKHWYHEVYDKAQKGFVEIEVDGVKISTYAQLQSLFEEAVSKDLAGMDDKNIKNHYQYTENLKWKIYKQLLKNTDGFSSDLFTAPQAGSG

SEQ ID NO: 4 - RNA, 1077 nt, Streptococcus pneumoniae
auggauggcauugcgaaagaauggggcuuugcgagccgcagcgcguauagccgcgauauucuggcgauggaaagcuuuagcaccuauauuggcaaccaguuuacccgcaccgaaaaacugcagacccagaccggcaacgauagcaugaaauuuaacuauaacggcauuagccaguuuagcagcgugcgcaaccgcagcagcagcagcacccuggauaaacugggcuuuaaaagcagcggcaccaaccugaaccugcgcuaugcgaacaacagcauucuggcggauagccuguuuggcauucaguauaacauuagcgauagcccgauugauaaauauggcuuuaaagauauuuaucagaaagauaaccugacccuguaugaaaaccaguauagccugccgauugcgguggcgagccagagcguguauaacgaugugaaauuuaacgaacauacccuggauaaccaggcgagcuuucugaaccagcuggcgaacgugaacuuugauuauuuuagcccgauuccguaugaaaaaaccgaaaaaauugaaaacaccaacgaucugauuagcgugaccagcagcagcaacgaagaugcggcgauucaguaucagauugaagugccggaaaacagccagguguaucugagcuuuauuaaccugcauuuuagcaacgauaaacagaaaaaaguggauauucuggugaacggcgaaaaaaaaaccuuuaccaccgauaacguguuuagcuuuuuuaaccugggcuauaccaaagaaaaaaaaaccuuuaacauuaacgugagcuuuccgggcaacagccaggugagcuuugaaagcccgaccuuuuaucgccuggauaccaaaaccuuuaccgaagcgauucagaaaauuaaagaacagccggugaccgugagcaccagcaaaaacaaaguguuugcgaccuaugaugugcagcaggauaccagcauuuuuuuuaccauuccguaugauaaaggcuggagcgcguaucaggauggcaaaaaaauugaaauuaaacaggcgcagaccggcuuuaugaaaguggauauuccgaaaggcggcagcggc

SEQ ID NO: 5 - RNA, 1662 nt, Streptococcus pneumoniae
augagcuuugauggcuuuuuucugcaucauauuguggaagaacugcgcagcgaacuggugaacggccgcauucagaaaauuaaccagccguuugaacaggaacuggugcugcagauucgcagcaaccgccagagccaucgccugcugcugagcgcgcauccgguguuuggccgcauucagcugacccagaccaccuuugaaaacccggcgcagccgagcaccuuuauuauggugcugcgcaaauaucugcagggcgcgcugauugaaagcauugaacagguggaaaacgaucgcauuguggaaauuaccgugagcaacaaaaacgaaauuggcgaucauauucaggcgacccugauuauugaaauuaugggcaaacauagcaacauucugcugguggauaaaagcagccauaaaauucuggaagugauuaaacaugugggcuuuagccagaacagcuaucgcacccugcugccgggcagcaccuauauugcgccgccgagcaccaaaagccugaacccguuuaccauuaaagaugaaaaacuguuugaaauucugcagacccaggaacugaccgcgaaaaaccugcagagccuguuucagggccugggccgcgauaccgcgaacgaacuggaacgcauucuggugagcgaaaaacugagcgcguuucgcaacuuuuuuaaccaggaaaccaaaccgugccugaccgaaaccagcuuuagcccggugccguuugcgaaccaggugggcgaaccguuugcgaaccugagcgaucugcuggauaccuauuauaaagauaaagcggaacgcgaucgcgugaaacagcaggcgagcgaacugauucgccgcguggaaaacgaacugcagaaaaaccgccauaaacugaaaaaacaggaaaaagaacugcuggcgaccgauaacgcggaagaauuucgccagaaaggcgaacugcugaccaccuuucugcaucaggugccgaacgaucaggaucaggugauucuggauaacuauuauaccaaccagccgauuaugauugcgcuggauaaagcgcugaccccgaaccagaacgcgcagcgcuauuuuaaacgcuaucagaaacugaaagaagcggugaaauaucugaccgaucugauugaagaaaccaaagcgaccauucuguaucuggaaagcguggaaaccgugcugaaccaggcgggccuggaagaaauugcggaaauucgcgaagaacugauucagaccggcuuuauucgccgccgccagcgcgaaaaaauucagaaacgcaaaaaacuggaacaguaucuggcgagcgauggcaaaaccauuauuuaugugggccgcaacaaccugcagaacgaagaacugaccuuuaaaauggcgcgcaaagaagaacugugguuucaugcgaaagauauuccgggcagccauguggugauuagcggcaaccuggauccgagcgaugcggugaaaaccgaugcggcggaacuggcggcguauuuuagccagggccgccugagcaaccuggugcagguggauaugauugaagugaaaaaacugaacaaaccgaccggcggcaaaccgggcuuugugaccuauaccggccagaaaacccugcgcgugaccccggauagcaaaaaaauugcgagcaugaaaaaaagcggcagcggc

SEQ ID NO: 6 - RNA, 3903 nt, Streptococcus pneumoniae
auggauaaacugaaauauuaucagggcuauacccugagcaccaccaugguguaugaucgcggcgaaggcgaagaaaccgaaaaacuggaagauaaacagauucagcuggaucugaaaaaaguggaaauuaaaaacauuaaagaaaccagccugaugaacguggaugcggaaggcaacgaaaccgauaaaagccugcugagcgaaaaaccgaccgaugugagccagcuguaucugcgcgugaccacccaugauaacaaagugacccgccuggcggugagcagcguggaagaaguggugguggauggcaaaacccuguauaaagugguggcgaaagcgccggaucuggugcagcgccgcgcggaugauacccugagcgaagaauaugugcauuauuuugaaaaacagcugccgaaagugaacaacguguauuauaacuuuaacgaacuggugaaagauaugcaggcgaacccgaugggcgaauuuaaacugggcgcggaucugaacgcggugaacgugaaaccggcgggcaaagcguaugugauggcgaaauuucgcggcacccugagcagcguggaaaaccaucaguauaccauucauaaccuggaacgcccgcuguuuaacgaagcggaaggcgcgacccugaaaaacuuuaaccugggcaacgugaacauuaacaugccgugggcggauaaaguggcgccgauuggcaacauguuuaaaaaaagcacccuggaaaacauuaaaguggugggcagcgugaccggcaacaacgaugugaccggcgcggugaacaaacuggaugaagcgaacaugcgcaacguggcguuuauuggcaaaauuaacagccugggcgauaaaggcugguggagcggcggccuggugagcgaaagcuggcgcagcaacaccgauagcguguauuuugauggcgauauugugggcaacaacagcaaauuuggcggccugguggcgaaagugaaccauggcagcaaccagugggaugugaaacagaaaggccgccugaccaacagcguggugaaaggcaccaugacccugaaaaaccauggccagagcggcggccuggugcaugaaaacuaugauuggggcuggguggaaaacaacauuagcaugaugaaagugaacaacggcgaaauuauguauggcagcggcagcauugauggcgauccguauuuuggcuuugauuauuuuaaaaacaacuauuaugugaaagauguggcgaccggcgaaagcaccuauaaacgcagcaaacagauucagagcauuagccaggcggaagcggaugcgaaaauugcgaacaugggcauuaccgcgaacaccuuugcgauucaggauccgguggugaacaaacugaaccgcauuauugaucgcgauagcgaauauaaagcgauucaggauuaucaggaaacccgcaaccuggcguaucgcaaccuggaaaaacugcagccguuuuauaacaaagaauggauugugaaccagggcaacaaacugaccgaugaaagcaaccuggugaaaaaaaccgugcugagcgugaccggcaugaaaagcggccaguuugugaccgaucugagcagcguggauaaaauuaugauucauuaugcggauggcaccaaagaagaauuuggcgugagcgcgauuagcgauagccgcgugaaacaggugaaagaauauaacguggaugaucugggcgugguguauaccccgaacaugguggauaaaaaccgcgauagccugauuaccaaagugaaagaaaaacugagcagcguggcgcuggauagcgcggaagugaaaagcauuaccaacaacccggcgagccuguaucuggaagaaagcuuugcggaagugcgcgaaacccuggauaaacuggugaaaagccugcuggaaaacgaagaucaucagcugaacagcgaugaaguggcggaaaaagcgcugcugaaaaaaguggaagauaacaaagcgaaaauuauucuggcgcugaccuaucugaaccgcuauuauggcauugauuaugauggccugaacuuuaaacaucugaugauguuuaaaccggauuuuuauggcaaaaccccgagcauucuggauuuucugauucgcauuggcagcgcggaaaaaaaccugaaaggcgaucgcagccuggaagcguaucgcgaagugauuggcggcaccauuggcaaaggcgaacugaacggccugcugggcuauaacaugcgccuguuuaccaaauauaccgaucugaacgauugguuuauucaugcggcgaaaaacguguaugugagcgaaccggaaaccaccaccgaagauuuuaaagauaaacgccaucgcauuuaugauggccugaacaacgaugugcauggccgcaugauucugccgcugcugaaccugaaaaaagcgcauauuuuugugauuagcaccuauaacaccauugcguuuagcagcuuugaaaaauauggcaaaaacaccgaagaagaacgcaacgcguauaaagcggaaauugaucgcguggcgaaagcgcagcagcgcuaucuggauuuuuggagccgccuggcgcugccgaaagugcgcaaccagcugcugaaaagccagaacagcgugccgaccccggugugggauaaccagguguaugugggccugggcggcgcgaaccgcaugggcuauggcgauggcggccgcguggugaccccggugcgcgaacuguuuggcccgaccgaucgcuggcaucagauuaacuggaacaugggcgcgauggcgaaaauuuaugaacgcccguggaaagaugaucagguguauuuuauggugaccaacaugauggaaccguuuggcauuagcgcguuuacccaugaaaccacccaugugaacgaucgcauggcguauuauggcggcgauuggcaucgcgaaggcaccgaucuggaagcguuugcgcagggcaugcugcagaccccggauaaaagcaccaccaacggcgaauauggcgcgcugggcauuaacauggcguaugaacgcaaaaacgauggcgaacagcuguauaacuaugauccggaaaaacuggauagccgcgaaaaaauugauagcuauaugaaaaacuauaacgaaagcaugaugaugcuggauuaucuggaagcgagcgcggugauucgccagaaccugagcgauaacagcaaaugguuuaaaaaaauggauaaagaauggcgcaccaacgcggaucgcaaccgccugauuggcgaaccgcaucagugggauaaacugcgcgaucugaccgaagaagaaaaaaaacugccgauugauagcauugauaaacugguggaaaacaacuuugugacccugcauggcaugccgaaaaacggccgcuaucgcaccgaaggcuuugauagcagcuaucagccggugaacaugauggcgggcguguuuggcggcaacaccagcaaaagcaccgugggcagcauuagcuuuaaacauaacgcguuucgcauguggggcuauuauggcuaugaaaacggcuuuauuccguaugugagcaacaaacugaaaggcgcggcgaacaaagaaaacaaaggccugcugggcgaugauuuuauuauuaaaaaagugagcaaaaaccaguuucagaaccuggaagaauggaaaaaacauugguaucaugaaguguaugauaaagcgcagaaaggcuuuguggaaauugaaguggauggcgugaaaauuagcaccuaugcgcagcugcagagccuguuugaagaagcggugagcaaagaucuggcgggcauggaugauaaaaacauuaaaaaccauuaucaguauaccgaaaaccugaaauggaaaauuuauaaacagcugcugaaaaacaccgauggcuuuagcagcgaucuguuuaccgcgccgcaggcgggcagcggc